(19) United States
(12) Patent Application Publication
(10) Pub. No.: US 2024/0058578 A1
(43) Pub. Date: Feb. 22, 2024

(54) CATHETER

(71) Applicant: Shuttle Catheters PC, Athens (GR)

(72) Inventor: Giannis STEFANIDIS, Nea Erythrea (GR)

(21) Appl. No.: 18/267,031

(22) PCT Filed: May 20, 2021
(86) PCT No.: PCT/GR2021/000031

§ 371 Date: Jun. 13, 2023

(30) Foreign Application Priority Data
Dec. 18, 2020 (GR) 20200100742

(51) Int. Cl. A61M 25/00; A61M 25/10; A61B 17/22

(52) U.S. Cl. CPC A61M 25/0082; A61M 25/1018; A61B 17/22; A61B 2017/22051

(57) ABSTRACT

The present invention refers to a catheter (1) comprising a catheter shaft (2), a flexible moving element (3) and a surgical tool (5) connected to the flexible moving element (3). The catheter shaft (2) extends along a longitudinal axis (500) and comprises a flexible moving element receiving lumen (21) defined by a catheter shaft wall (24). The flexible moving element (3) is arranged in the flexible moving element receiving lumen (21) in such a way that the flexible moving element (3) is at least circumferentially supported by the catheter shaft wall (24) so that a movement of the flexible moving element (3) in a direction from a proximal shaft end (29) of the catheter shaft (2) to a distal shaft end (28) of the catheter shaft (2) causes a movement of the surgical tool (5).

The present invention refers to a catheter comprising a surgical tool. The catheter of the present invention can also be characterized as a medical catheter.

Catheters have been used for years in many medical applications for humans and animals, as they provide access to the body in a minimally invasive way.

For example, catheters are widely used in most endovascular applications. Due to their minimal invasive character, the treatment of blood vessel pathologies can be achieved without considerable postoperative pain and the need of a general anaesthesia during the operation or a long hospitalization of the patient. An endovascular catheter is known from WO 2016 203277 A1.

It is an object underlying the present invention to suggest a compact catheter with a high design flexibility and ease of use and manufacture, which can be used in a variety of medical applications.

The solution to said object is achieved by a catheter with the combination of features of the independent claim. The dependent claims contain advantageous embodiments and aspects of the invention.

In particular, the catheter comprises a catheter shaft, a flexible moving element and a surgical tool connected to the flexible moving element. The catheter shaft extends along a longitudinal axis and comprises a flexible moving element receiving lumen defined by a catheter shaft wall.

The flexible moving element is arranged in the flexible moving element receiving lumen in such a way that the flexible moving element is at least partially circumferentially supported by the catheter shaft wall so that a movement of the flexible moving element in a direction from a proximal shaft end of the catheter shaft to a distal shaft end of the catheter shaft causes a movement of the surgical tool.

In particular, the flexible moving element is arranged in the flexible moving element receiving lumen in such a way that the flexible moving element is at least partially circumferentially supported by the catheter shaft wall so that the surgical tool is pushable, more specifically movable in the direction from the proximal shaft end of the catheter shaft to the distal shaft end of the catheter shaft, by the flexible moving element.

In other words, the flexible moving element is particularly arranged in the flexible moving element receiving lumen in a way that it is laterally supported by the catheter shaft wall so that the flexible moving element, even though it is itself bendable, can move, in particular push, the surgical tool. This means that, should not have been for the circumferential support of the flexible moving element provided by the catheter shaft wall, the flexible moving element would, at least partially, bend when being moved, in particular pushed, and thus would not be able to transmit said movement to the surgical tool. To put it differently, the flexible moving element is advantageously confined by the catheter shaft wall so that the flexible moving element functions as a stiffer element when being moved, in particular pushed, so that the flexible moving element can transmit its movement to the surgical tool. Thus, the catheter shaft wall acts as a restriction and support element for the flexible moving element so that the flexible moving element is supportable throughout its movement, in particular pushing movement, such that its movement results in a corresponding movement of the surgical tool, which is connected thereto, i.e. its movement is such that the surgical tool can be moved, in particular pushed. In other words, the catheter shaft wall provides to the flexible moving element the stiffness that the flexible moving element inherently lacks and thus needs in order to be able to move the surgical tool forward. It is understood that the flexible moving element acts as a movement and force transmitting means that is configured to transmit a force applied to the flexible moving element and thus a movement to the surgical tool. At the same time, the flexible moving element is/remains capable of being bent, when the catheter, in particular the catheter shaft, is forwarded through curved paths, e.g. tortuous blood vessels, of the body of a patient.

The catheter of the present invention has a compact structure, as due its suggested configuration it is sufficient that the catheter shaft comprises only one lumen for receiving a means for transmitting a force and thus a movement to the surgical tool. In the present invention, said means corresponds to the flexible moving element being arranged in the flexible moving element receiving lumen. More specifically, the present invention allows for a reduction of a maximum outer dimension, in particular a diameter, of the catheter/catheter shaft by about 30% compared to a catheter with a catheter shaft requiring two lumens for receiving the means for transmitting a movement to the surgical tool. This in turn has the advantage that the catheter can be used in a large variety of applications and more particularly in applications where blood vessels, lumens and cavities of small dimensions of a human or animal body should be accessed. Further, due to the flexible nature of the moving element the catheter can still be bent as a whole, while the catheter is provided with a mobile surgical tool that is movable relative to the catheter shaft. Thus, the flexible moving element does not alter the flexible nature of the catheter as a whole. It is noted that in the framework of the present invention, a catheter is in particular understood as a device having a flexible (catheter) shaft for insertion into a body cavity, duct, canal, vessel or passageway of a patient and being configured to deliver to or withdraw fluids from a patient through its catheter shaft and/or distend a passageway and/or place a surgical tool at a target site inside the patient's body in order to cause a modification of the body cavity, duct, canal, vessel or passageway.

Continuing with the advantages of the catheter of the present invention, the fact that only one lumen is needed for accommodating the arrangement of the flexible moving element offers a great flexibility in view of the spatial arrangement of the lumen with respect to any further lumens that might be formed in the catheter shaft, such as a lumen for transferring fluids therethrough and/or receiving a guidewire. More specifically, the requirement of only one lumen in the catheter shaft of the catheter of the present invention for receiving the means for moving the surgical tool forward is particularly advantageous, when a further lumen is formed in the catheter shaft for transferring fluids, e.g. body fluids, therethrough, and said further lumen needs to have a large cross-section in order to facilitate the transfer of large fluid quantities. That is because the single lumen for the means for moving the surgical tool, namely the already described flexible moving element receiving lumen, leaves free space in the catheter shaft so that the further lumen can be formed with the necessary size.

Further, as the surgical tool can be moved by the flexible moving element provided in a single lumen, said flexible moving element can have a small length, in particular compared to a catheter shaft that requires two lumens for receiving a means for transmitting a movement to the surgical tool. Thereby, friction losses during the movement of the flexible moving element inside the flexible moving element receiving lumen can be reduced and the risk of a potential warping of the catheter shaft can also be reduced or even eliminated.

In addition, the suggested catheter does not require a complex manufacturing process, while it also enables a simple and intuitive handling by a doctor. The latter is achieved as a pushing force, i.e. a force applied by the doctor to flexible moving element towards the distal shaft end of the catheter shaft, is translated into a movement of the surgical tool in the same direction, namely towards the distal shaft end. Accordingly, a pulling force, i.e. a force applied by the doctor to the flexible moving element towards the proximal shaft end of the catheter shaft is translated into a movement of the surgical tool in the same direction, namely towards the proximal shaft end.

It is noted that in the expression “at least partially circumferentially supported” the term “at least partially” particularly refers to the term “circumferentially”. This means that the flexible moving element is supported over a part of its whole circumference or its whole circumference at a given cross-section. In particular, in the framework of the present invention, the flexible moving element is partially or completely circumferentially supported inside the flexible moving element receiving lumen regardless of the form/shape of the inner surface of the catheter shaft wall defining the flexible moving element receiving lumen or in other words regardless of the form/shape of the flexible moving element receiving lumen, when/as long as the flexible moving element moves substantially or only along its longitudinal axis and does not move or bend laterally. A partial circumferential support is particularly provided when there is a partial circumferential contact between the flexible moving element and the flexible moving element receiving lumen, whereas a complete circumferential support is particularly provided when there is a complete circumferential contact between the flexible moving element and the flexible moving element receiving lumen.

The inner surface of the catheter shaft wall defining the flexible moving element receiving lumen may preferably be configured such that it provides the at least partial circumferential support over only a portion of its length or over its whole length to the flexible moving element.

Preferably, the flexible moving element is substantially arranged in the flexible moving element receiving lumen. “Substantially” means in particular that more than 50%, preferably more than 60%, more preferably more than 70%, even more preferably more than 80% of a total length of the flexible moving element or of a length of the flexible moving element that is always arranged inside the catheter shaft.

Preferably, the flexible moving element is completely circumferentially supported by the catheter shaft wall. This means that the flexible moving element is supported over its whole circumference at a given cross-section. The complete circumferential support can take place over only a part or the whole length of the flexible moving element.

Advantageously, the catheter shaft comprises a distal shaft wall opening that is formed in a distal circumferential area of the catheter shaft and communicates with the flexible moving element receiving lumen. In an embodiment of the present invention, the flexible moving element is configured to move the surgical tool out of the catheter shaft through the distal shaft wall opening. In another embodiment of the present invention, a portion of the flexible moving element is (advantageously always, i.e. during use or non-use of the catheter and in any position of the flexible moving element with regard to the flexible moving element receiving lumen) located in the distal shaft wall opening.

The distal shaft wall opening can advantageously be formed as a recess in the catheter shaft wall, in particular as a through recess in a transverse direction of the catheter shaft.

Advantageously, the flexible moving element is longitudinally movably arranged in the flexible moving element receiving lumen. More particularly, the flexible moving element is extractably arranged in the flexible moving element receiving lumen. This means that the flexible moving element can be moved out of the flexible moving element receiving lumen due to the at least partial circumferential support by the catheter shaft wall so that the flexible moving element can move the surgical tool.

Advantageously, the surgical tool and the flexible moving element each have two end positions. In the first end position (proximal end position), a distal end of each of these components is at its furthest position with respect to the distal shaft end of the catheter shaft, whereas in the second end position (distal end position) the distal end of each of these components is at its closest position with respect to the proximal shaft end of the catheter shaft. Thus, each of these components is movable between its corresponding first end position and its second end position. The difference between the first end position and the second end position of the surgical tool corresponds to the range of motion of the surgical tool, in other words the maximum distance that the surgical tool may cover during its movement. In particular, the flexible moving element is extracted in its second end position and retracted in its first end position.

The flexible moving element is advantageously flexible over its whole length. Furthermore, a flexibility of the flexible moving element may vary along its length. This allows the flexible moving element to be adapted to a respective configuration and/or application of the catheter.

According to an advantageous embodiment of the present invention, a distal portion and/or a proximal portion of the flexible moving element has/have a smaller flexibility than its middle portion, i.e. the portion between the distal portion and the proximal portion of the flexible moving element. This is in particular of advantage, when the surgical tool is directly connected to the flexible moving element. The distal portion having a smaller flexibility than the middle portion may correspond to the portion of the flexible moving element that is outside the flexible moving element receiving lumen, when the flexible moving element is in its second end position. The distal portion and/or the proximal portion of the flexible moving element preferably correspond to connecting portions of the flexible moving element with other components of the catheter.

Preferably, the flexibility of the flexible moving element is such that a flexibility of the catheter shaft and/or the catheter with the flexible moving element is not more than 20%, preferably not more than 10%, most preferably not more than 5%, different, in particular larger, than a flexibility of the catheter shaft and/or the catheter without the flexible moving element. Most preferably, the flexibility of the flexible moving element is substantially the same with the flexibility of the catheter shaft.

In the framework of the present invention, the term “flexible” describing a component, in particular the moving element, means advantageously that the respective component, in particular the moving element, is bendable by its own weight. In other words, the term “flexible” means that the respective component, in particular the flexible moving element, cannot support/carry/bear its own weight without being bent when fixed at one of its ends. It is noted that bending does not mean that the respective component collapses due its own weight when it is fixed at one of its ends. In other words, the flexible component may only partially carry its own weight.

Advantageously, the surgical tool is connected to the flexible moving element at a distal portion and/or distal end of the flexible moving element.

According to an advantageous embodiment of the present invention, a distal portion and/or a proximal portion of the flexible moving element has/have a smaller flexibility than a middle portion, i.e. the part between the distal portion and the proximal portion of the flexible moving element. This is in particular of advantage, when the surgical tool is directly connected to the flexible moving element and can freely exit through a shaft wall opening of the catheter.

It is preferred that at least the flexible moving element portion that is movably arranged or arrangeable/movable in the flexible moving element receiving lumen between the first and the second end positions of the flexible moving element has a cross-sectional area over at least a part of its whole length, i.e. over only a part of its whole length or over its whole length, that is at least 80%, more preferably at least 85%, even more preferably at least 90%, even more preferably at least 95%, most preferably substantially 100%, of a cross-sectional area of the flexible moving element receiving lumen.

Most preferably, the flexible moving element has over its whole length a cross-sectional area that is at least 80%, more preferably at least 85%, even more preferably at least 90%, even more preferably at least 95%, most preferably substantially 100%, of a cross-sectional area of the flexible moving element receiving lumen. Advantageously, a fit between the flexible moving element and the lumen is a clearance fit.

In particular, when the flexible moving element receiving lumen and at least the flexible moving element portion that is movably arranged or arrangeable/movable in the flexible moving element receiving lumen between the first and second end positions of the flexible moving element are each formed as a circular cylinder, it is preferred that at least said flexible moving element portion has over at least a part of its whole length, i.e. over only a part of its whole length or over its whole length, a diameter that is at least 80%, more preferably at least 85%, even more preferably at least 90%, even more preferably at least 95%, most preferably substantially 100%, of a diameter of the flexible moving element receiving lumen.

Most preferably, when the flexible moving element receiving lumen and the whole flexible moving element are each formed as a circular cylinder, it is preferred that the flexible moving element has over its whole length a diameter that is at least 80%, more preferably at least 85%, even more preferably at least 90%, even more preferably at least 95%, most preferably substantially 100%, of a diameter of the flexible moving element receiving lumen.

It is noted that when referring to the shape of a portion or the whole of the flexible moving element, the shape of a main body of the flexible moving element that extends in the direction of its longitudinal axis, without taking into account any potential protrusions that may act as connecting portions of the flexible moving element with a further component of the catheter, i.e. when the flexible moving element substantially extends along its longitudinal axis, is in particular meant within the scope of the present invention. Thus, a length of a portion or the whole of the flexible moving element means in particular a length of the portion or the whole of the flexible moving element in the direction of its longitudinal axis, respectively.

It is further noted that the term “at least the flexible moving element portion” means in particular the portion of the flexible moving portion, that is arranged in the flexible moving element receiving lumen during its movement from the first end position to the second end position or vice versa.

In particular, said flexible moving element portion has a length that is the sum of the length of the flexible moving element receiving lumen and a length that corresponds to the maximal distance that the surgical tool can be moved between the first end position and the second end position of the surgical tool. For example, if the flexible moving element receiving lumen has a length of 20 cm and the surgical tool can be moved by a maximum of 2 cm, said flexible moving element portion has a length of 22 cm. In other words, said flexible moving element portion corresponds to the portion of the flexible moving element that is arranged in the flexible moving element receiving lumen and the distal portion of the flexible moving element located outside the flexible moving element receiving lumen, when the flexible moving element is in its second end position.

In case the flexible moving element receiving lumen is interrupted by at least one further shaft wall opening provided between the proximal shaft wall opening and the distal shaft wall opening, then said flexible moving element portion particularly corresponds to the portion of the flexible moving element that is arranged in the flexible moving element receiving lumen, the portion of the flexible moving element located in the at least one further shaft wall opening provided between the proximal shaft wall opening and the distal shaft wall opening, and the distal portion of the flexible moving element located outside the flexible moving element receiving lumen, when the flexible moving element is in its second end position. In this case, said flexible moving element portion has a length that is the sum of the length of the flexible moving element receiving lumen, the length of the at least one further shaft wall opening and a length that corresponds to the maximum distance that the surgical tool can be moved between the first end position and the second end position of the surgical tool. In other words, said flexible moving element portion has a length that is the sum of the length of the flexible moving element receiving lumen as if it were not interrupted by the at least one further shaft wall opening and a length that corresponds to the maximum distance that the surgical tool can be moved between the first end position and the second end position of the surgical tool.

The length of said flexible moving element portion means in particular a length of said flexible moving element portion in the direction of its longitudinal axis.

By choosing the dimensional relationship of the flexible moving element and the flexible moving element receiving lumen as presented, it can be ensured that most of the force with which the flexible moving element is moved, in particular pushed, can be transferred to the surgical tool and cause it to move, in particular be pushed.

The term “substantially” in connection with the terms “cross-sectional area” and/or “diameter” means in particular that the flexible moving element receiving lumen and the flexible moving element are produced within the technical tolerances of the method(s) used to manufacture said elements.

Preferably, at least the flexible moving element portion that is movably arranged or arrangeable/movable in the flexible moving element receiving lumen between the first and the second end positions of the flexible moving element has over at least a part of its whole length, i.e. over only a part of its whole length or over its whole length, the same shape with the flexible moving element receiving lumen. Most preferably, the flexible moving element has over its whole length the same shape with the flexible moving element receiving lumen.

Preferably, the flexible moving element is arranged in the flexible moving element receiving lumen in such a way that the flexible moving element portion that is movably arranged or arrangeable/movable in the flexible moving element receiving lumen between the first and the second end positions of the flexible moving element has over at least a part of its whole length, i.e. over only a part of its whole length or over its whole length, a shape that is complementary to a shape of the flexible moving element receiving lumen. For example, if both the flexible moving element and the flexible moving element receiving lumen have an elliptical cross-section, the flexible moving element is arranged in the flexible moving element receiving lumen in such a way that a major axis and a minor axis of the flexible moving element are aligned with a major axis and a minor axis of the flexible moving element receiving lumen, respectively.

According to an advantageous embodiment, at least the flexible moving element portion that is movably arranged or arrangeable/movable in the flexible moving element receiving lumen between the first and the second end positions of the flexible moving element, in particular the whole flexible moving element, is formed as a circular cylinder.

Preferably, the flexible moving element receiving lumen is formed as a circular cylinder.

Preferably, the catheter shaft has a cylindrical shape, in particular with a circular cross-section, i.e. the catheter shaft is in particular a circular cylinder. However, the cross-section of the cylindrical catheter shaft may also have another shape, e.g. the cross-section may be oval. A cylindrical catheter shaft is easy to be manufactured.

According to an alternative advantageous embodiment of the present invention, the catheter shaft may comprise at least one frustoconical region, in particular a plurality of frustoconical regions. In addition to the at least one frustoconical region, the catheter shaft may also comprise at least one cylindrical region. Forming the catheter shaft with at least one frustoconical region has the advantage that a sliding friction between the catheter shaft and the wall of a body lumen, inside which the catheter may be pushed forward, or between the catheter shaft and a casing arranged on the catheter shaft and having a different shape than the catheter shaft, can be reduced due to the smaller contact between the catheter shaft and the lumen or the catheter shaft and the casing, respectively.

According to an advantageous embodiment of the present invention, the flexible moving element receiving lumen extends from aproximal shaft end of the catheter shaft in a direction towards a distal shaft end of the catheter shaft. This means in other words that a proximal end of the flexible moving element receiving lumen coincides with a proximal shaft end of the catheter shaft. In this case, the flexible moving element may exit through an opening at the proximal end of the flexible moving element receiving lumen, so that it can be manipulated by the doctor. It is noted that the formulation “in a direction towards a distal shaft end of the catheter shaft” does not mean that the flexible moving element receiving lumen extends to, i.e. reaches, the distal end of the catheter, but indicates the direction in which the flexible moving element extends.

According to an alternative advantageous embodiment of the present invention, a proximal end of the flexible moving element receiving lumen is spaced apart from a proximal shaft end of the catheter shaft. In other words, the flexible moving element receiving lumen according to this embodiment extends from a (proximal) region of the catheter shaft spaced apart from the proximal shaft end of the catheter shaft in a direction towards a distal shaft end of the catheter shaft, and not from the proximal shaft end of the catheter shaft. In order to control the flexible moving element in this embodiment, a proximal shaft wall opening may advantageously be formed in a proximal circumferential area of the catheter shaft that communicates with the flexible moving element receiving lumen. The flexible moving element can thus be controlled via the proximal shaft wall opening. The proximal shaft wall opening can advantageously be formed as a recess in the catheter shaft wall, in particular a through recess in a transverse direction of the catheter shaft.

In the framework of the present invention, the longitudinal axis of the catheter shaft advantageously corresponds to a longitudinal axis of the catheter. Further, a proximal shaft end and a distal shaft end of the catheter shaft advantageously correspond to a distal end and a proximal end of the catheter, respectively.

In general, the term “proximal” describing a feature of the catheter or of any of its components means that said feature is closer to the doctor than a distal feature, when the catheter is used on a patient or held with the appropriate orientation corresponding to its intended use, i.e. the insertion into the patient, whereas the term “distal” means that said feature is further away from the doctor compared to the proximal feature. For example, a proximal end of the catheter is closer to the doctor than a distal end of the catheter. In particular, the respective proximal feature may advantageously be located outside the patient's body, while the respective distal feature may be located inside the patient's body, when the catheter is (already) inserted in the patient in view of its intended use. For example, the distal shaft wall opening is located inside the patient's body during use of the catheter. Accordingly, when the catheter shaft has a proximal shaft wall opening, the proximal shaft wall opening is advantageously located outside of the patient's body during the catheter use. Specifically, the term “proximal area/region” with regard to the catheter or any of its components means in particular that this area is located between a proximal end of the catheter or the corresponding component and a middle of the catheter or the corresponding component, respectively, in the direction of its respective longitudinal axis. Accordingly, the term “distal area/region” with regard to the catheter or any of its components may in particular mean that this area/region is located between a middle of the catheter or the corresponding component until the distal end of the catheter or the corresponding component, respectively, in the direction of its respective longitudinal axis.

According to an embodiment of the present invention, the surgical tool can be directly connected to the flexible moving element. It is understood by the term “directly” that no other component of the catheter (except for any potential joining means needed to join the flexible moving element to the surgical tool) is arranged between the surgical tool and the flexible moving element at the connection point.

According to an embodiment, the surgical tool may advantageously be indirectly connected to the flexible moving element. It is understood by the term “indirectly” that one or more components, preferably only one component, of the catheter (apart from any potential joining means needed to join the flexible moving element to the surgical tool) can be arranged between the surgical tool and the flexible moving element at the connection point.

The surgical tool can particularly be longitudinally movably, in particular slidably, arranged on the catheter shaft.

In particular, the surgical tool can be movably arranged directly on the catheter shaft (catheter shaft wall). When the surgical tool is an inflatable balloon, the inflatable balloon can for example have a form that partly or completely surrounds the catheter shaft. For example, the inflatable balloon may be formed as a donut, a hollow cylinder or a hollow barrel.

Alternatively, the surgical tool can be longitudinally movably, in particular slidably, arranged on the catheter shaft by being arranged on a further component of the catheter shaft.

In particular, the catheter preferably comprises a tool-receiving casing that is longitudinally movably arranged on the catheter shaft and connected to the flexible moving element, wherein the surgical tool is arranged on the tool-receiving casing. The tool-receiving casing is thus arranged between the flexible moving element and the surgical tool. In particular, the surgical tool is connected to the tool-receiving casing so that there is no relative movement between the surgical tool and the tool-receiving casing in the direction of the longitudinal axis of the tool-receiving casing. It is noted that the tool-receiving casing is advantageously fixed to the flexible moving element so that there is no relative movement between the flexible moving element and the tool-receiving casing at their connection point. The described arrangement of the tool-receiving casing, the catheter shaft and the flexible moving element with respect to each other is such that a movement of the flexible moving element is transmittable to the tool-receiving casing and in turn to the surgical tool arranged on, in particular fixed to, the tool-receiving casing. Thus, the surgical tool can be moved parallel to the longitudinal axis of the control shaft relative to the control shaft.

According to an embodiment of the present invention, the tool-receiving casing is preferably slidably arranged on the catheter shaft.

A longitudinal axis of the tool-receiving casing preferably corresponds to or is parallel to the longitudinal axis of the catheter shaft.

Preferably, at least a portion of the tool-receiving casing has an open cross-section. This means that the tool-receiving casing may preferably have an open cross-section over a portion thereof in a direction of its longitudinal axis or over its whole length. This is advantageous in particular when the surgical tool should be moved through narrow passages and tortuous lumens in the patient's body.

Advantageously, the open cross-section is such that the tool-receiving casing preferably substantially surrounds the catheter shaft. “Substantially” means in particularly that at least 60%, preferably at least 70%, more preferably at least 80%, of a corresponding cross-section of the catheter shaft is surrounded by the corresponding open cross-section of the tool-receiving casing. Especially when the open cross-section of the tool-receiving casing has a circular or oval form, “substantially” can additionally or alternatively mean that the cross-section may extend over at least 220 degrees, more preferably over at least 260 degrees, even more preferably over at least 290 degrees. This has the advantage that the tool-receiving casing cannot be demounted from the catheter shaft even if the catheter shaft is being forwarded through narrow and tortuous passages of the patient's body.

Alternatively, the tool-receiving casing may have a closed cross-section over its whole length.

The tool-receiving casing is advantageously arranged on the catheter shaft so that the tool-receiving casing partially or completely covers the distal shaft wall opening. Thus, the tool-receiving casing provides further support to the flexible moving element. Particularly in the case of the tool-receiving casing partially covering the distal shaft wall opening, it can be of advantage if the flexibility of a distal portion of the flexible moving element is chosen such that the flexible moving element is adequately supported at its distal portion. In particular, the flexibility of the distal portion of the flexible moving element can be of lower flexibility than the middle portion of the flexible moving element.

The dimension of the distal shaft wall opening parallel to the longitudinal axis of the catheter shaft advantageously substantially corresponds to the distance that the tool receiving casing and preferably the surgical tool can be moved parallel to the direction of the longitudinal axis of the catheter shaft. The term “substantially” is used here to denote that a part of the flexible moving element connecting to the tool-receiving casing is preferably arranged in the distal shaft wall opening. Thus, the distance that the tool-receiving casing can be moved is equal to the dimension of the distal shaft wall opening parallel to the longitudinal axis of the catheter shaft reduced by the dimension of the distal part of the flexible moving element arranged in the distal shaft wall opening in the direction parallel to the longitudinal axis of the catheter shaft.

According to an advantageous embodiment of the present invention, the tool-receiving casing may have the same outer shape with the (outer) shape of the catheter shaft and/or a complementary inner shape to the (outer) shape of the catheter shaft.

Preferably, an outer shape and an inner shape of the tool-receiving casing are the same.

Preferably, the tool-receiving casing has a hollow cylindrical shape, in particular with a circular cross-section, i.e. the tool-receiving casing is in particular a hollow circular cylinder. However, the cross-section of the tool-receiving casing may also have another shape, e.g. the cross-section may be oval.

Most preferably, the tool-receiving casing is a hollow circular cylinder and the catheter shaft a circular cylinder. In this case, a fit between the tool-receiving casing and the catheter shaft is advantageously a clearance fit, which means that an inner diameter of the tool-receiving casing is larger than an outer diameter of the catheter shaft.

According to an alternative advantageous embodiment of the present invention, an inner shape of the tool-receiving casing and a(n) (outer) shape of the catheter shaft are different. This results in a smaller contact between the tool-receiving casing and the catheter shaft compared to the case where the tool-receiving casing and the catheter shaft have complementary shapes.

With this configuration, friction losses during the movement of the tool-receiving casing and the catheter shaft relative to each other can be reduced. Preferably, the tool-receiving casing is formed as a hollow circular cylinder, while the catheter shaft comprises at least one frustoconical region.

Preferably, the tool-receiving casing is further rotatably arranged on the catheter shaft. This allows the surgical tool not only to be movable parallel to the longitudinal axis of the catheter shaft but also rotatable around its longitudinal axis. This enhances the flexibility of the catheter in terms of placing the surgical tool at the desired position and with the desired orientation within the body of the patient. Additionally, the arrangement of the tool-receiving casing makes it easier for the tool-receiving casing and thus the surgical tool to be moved through narrow passages in the patient's body. When the surgical tool is an inflatable balloon and the balloon is inflated and anchored to a body lumen, the rotation of the tool-receiving casing and thus of the balloon is transformed to a rotation of the catheter shaft, which can thus be forwarded more easily through a narrow passage of the lumen. In order for the tool-receiving casing to be rotated, the flexible moving element is preferably rotatably arranged inside the flexible moving element receiving lumen. In view of this, the distal shaft wall opening is formed such that a rotation of the flexible moving element around the longitudinal axis of the flexible moving element is possible.

Most preferably, the tool-receiving casing is rotatably arranged on the catheter shaft such that a rotational range of the tool-receiving casing and thus of the surgical tool comprises (up to) 180 degrees. Accordingly, the flexible moving element preferably has a rotational range of (up to) 180 degrees.

In particular, the tool-receiving casing and thus the surgical tool can be configured to rotate (up to) 90 degrees in both directions around the longitudinal axis of the tool-receiving casing, wherein the longitudinal axis of the tool-receiving casing lies in a symmetry plane of the catheter shaft. Accordingly, the flexible moving element can preferably be configured to rotate (up to) 90 degrees in both directions around its longitudinal axis, wherein the longitudinal axis of the flexible moving element lies in a symmetry plane of the catheter shaft. The “0 degrees”-position of the tool-receiving casing or of the flexible moving element corresponds to the symmetry plane of the catheter shaft. To this end, it is preferred that the distal shaft wall opening is a formed as a recess that goes through the catheter shaft wall in a transverse axis of the catheter shaft. The transverse axis is vertical to the longitudinal axis of the catheter shaft. When the catheter shaft is formed as a circular cylinder, the transverse axis can in particular be a radial axis.

In order to control the movement of the surgical tool, the catheter preferably comprises a control handle that is connected to the flexible moving element. In other words, the control handle is fixed to the flexible moving element so that the control handle cannot move relative to the flexible moving element at their connection point. With this arrangement, a movement of the control handle is transmittable to the flexible moving element and in turn to the surgical tool. The control handle has a first (proximal) end position and a second (distal) end position. In the first end position, a distal end of the control handle is at its furthest position from the distal shaft end of the catheter shaft. In the second end position, a distal end of the control handle is at its closest position to the distal shaft end of the catheter shaft. The first end position and the second end position of the stiff control element correspond to the first end position and the second end position of the surgical tool, respectively.

It is a preferred embodiment that the control handle comprises a control casing that is longitudinally movably, in particular slidably, arranged on the catheter shaft and connected to the tool-receiving casing. In particular, the control casing can be directly connected to the flexible moving element and thus connected to the tool-receiving casing via the flexible moving element. Alternatively, the control handle may further comprise a stiff control element that is arranged between and connected to the flexible moving element and the control casing. The stiff control element is advantageously arranged inside the flexible moving element receiving lumen throughout its movement between its first and second position.

According to an alternative advantageous embodiment of the present invention, the control handle may comprise only the stiff control element. The stiff control element is connected to the flexible moving element. Thus, the surgical tool can be controlled by manipulating the stiff control element. In particular, the stiff control element can be formed for example as a shaft, grip button or the like. The stiff control element can advantageously be movable inside the flexible moving element receiving lumen between its first end position and its second end position. In particular, the stiff control element is partially arranged inside the lumen throughout its complete movement between its first end position and second end position.

In the case that the control handle comprises the stiff control element, it is noted that the stiff control element is always (during its movement between its first and second end position) arranged outside of the patient's body.

In the framework of the present invention, the term “stiff” describing the control element means that the control element is not bendable by its own weight. In other words, the term “stiff” means that the control element can support/carry/bear its own weight without being bent when it is fixed at one of its ends and the other end is free.

In an advantageous manner, the control casing may have the same outer shape with the (outer) shape of the catheter shaft and/or a complementary inner shape to the (outer) shape of the catheter shaft.

Preferably, an outer shape and an inner shape of the control casing are the same.

Preferably, the control casing has a hollow cylindrical shape, in particular with a circular cross-section, i.e. the control casing is in particular a hollow circular cylinder. However, the cross-section of the control casing may also have another shape, e.g. its cross-section may be oval.

According to a preferred embodiment of the present invention, the control casing is a hollow circular cylinder and the catheter shaft a circular cylinder. In this case, a fit between the control casing and the catheter shaft is advantageously a clearance fit, which means that an inner diameter of the casing is larger than an outer diameter of the catheter shaft.

According to an alternative advantageous embodiment of the present invention, an inner shape of the control casing and a(n) (outer) shape of the catheter shaft are different. This results in a smaller contact between the control casing and the catheter shaft compared to the case where the inner shape of the control casing and the (outer) shape of the catheter shaft are the same. Thus, friction losses during the relative movement between the control casing and the catheter shaft can be reduced. Preferably, the control casing is formed as a hollow circular cylinder, while the catheter shaft comprises at least one frustoconical region.

The control casing can also be rotatably arranged on the catheter shaft.

Instead of having the tool-receiving casing and the control handle, in particular the control casing, as separate components, the tool-receiving casing can be formed such that the tool-receiving casing also serves as a control handle for controlling the movement of the surgical tool. It is pointed out that, though the tool-receiving casing also serves as the control handle, it is the flexible moving element that transmits the movement to the surgical tool, and not the tool-receiving casing, when the tool-receiving casing is being moved. Otherwise, should not have been for the flexible moving element, the tool-receiving casing being itself flexible would tend to warp, when being pushed forward with respect to the catheter shaft.

Using an alternative formulation to describe the embodiment, according to which there is only one casing acting both as the tool-receiving casing and the control casing, it could be said that the control casing and the tool-receiving casing are made in/as one piece/component or that the control casing can be integrated with the tool-receiving casing.

The tool-receiving casing when also serving as a control handle or the control casing as a separate component is arranged on the catheter shaft such that it partially or completely covers the proximal shaft wall opening. Thus, further support can be provided to the flexible moving element. Particularly in the case of the tool-receiving casing partially covering the proximal shaft wall opening, it can be of advantage if the flexibility of a proximal portion of the flexible moving element is chosen such that the flexible moving element is adequately supported at its proximal portion. In particular, the flexibility of the proximal portion of the flexible moving element can be of lower flexibility than the middle portion of the flexible moving element.

According to an advantageous embodiment of the present invention, the tool-receiving casing and the catheter shaft are threadably engaged with each other. Thus, the tool-receiving casing can be moved in the direction of the longitudinal axis of the catheter shaft in a controllable way due to the threaded engagement of the tool-receiving casing and the catheter shaft with each other. More specifically, the tool-receiving casing is internally threaded, while the catheter shaft is externally threaded. Here, the internal (inner) threads of the tool-receiving casing are in engagement with the external (outer) threads of the catheter shaft. In the case of the tool-receiving casing being formed such that it also acts as a control handle, a distal portion of the tool-receiving casing is preferably threaded.

In particular, the internal threads of the tool-receiving casing and the external threads of the catheter shaft are formed such that a movement of the control handle in the direction of its longitudinal axis causes a rotation of the tool-receiving casing around its longitudinal axis combined with a movement of the tool-receiving casing in the direction of its longitudinal axis, i.e. a helical movement of the tool-receiving casing. This configuration has the advantage that the force with which the control casing is being moved in the direction of its longitudinal axis is translated into a helical movement of the tool-receiving casing. It is understood that, when the threaded tool-receiving casing is formed such that it also acts as a control handle, a movement of the tool-receiving casing in the direction of its longitudinal axis by applying a corresponding force at its proximal end or area will eventually force the user of the catheter to rotate together with the tool-receiving casing.

The catheter shaft can be left-handed threaded or right-handed threaded. Accordingly, the tool-receiving casing can be left-handed threaded or right-handed threaded.

It is also possible that the catheter shaft comprises at least one region with a left-handed thread and at least one region with a right-handed thread. The tool-receiving casing is then formed accordingly. This configuration is advantageous as the surgical tool can be moved through narrow passages of different morphologies by turning the surgical tool counterclockwise and clockwise so that it goes round/avoids any potential obstacle in the path (e.g. plaque in a blood vessel, protrusion due to the anatomy of the path). When the surgical tool is an inflatable balloon and the balloon is inflated and anchored to a body lumen, the rotation of the tool-receiving casing and thus of the balloon counterclockwise and clockwise is transformed to a corresponding rotation of the catheter shaft, what allows the catheter shaft to be squeezed and forwarded through a narrow passage of the lumen.

According to an advantageous embodiment, the flexible moving element is formed as a wire. The term “wire” may also comprise a cable or any other flexible rod-shaped element. In another advantageous embodiment, the flexible moving element is formed as a sheet. The sheet can in particular be curved. However, the flexible moving element can be any element that can be bent, so that the catheter can be bent as a unit, when being forwarded through lumens and cavities of hollow organs of a patient's body.

The wire or the sheet may be made for example from metal, plastic or a composite material.

According to a preferred embodiment of the present invention, the surgical tool of the catheter is an inflatable balloon. With the help of the inflatable balloon, the suggested catheter acquires greater pushability, since the needed propulsive force for moving the catheter shaft forward can be applied close to its tip. The term “pushability” means the degree in which the force transmitted from a proximal end of the catheter is translated into movement of a catheter's distal end (catheter tip), which depends on the transmission of the force along the body of the catheter, is understood. In other words, the term “pushability” means the ease of advancing the catheter inside a lumen, e.g. a blood vessel, and is indicative of the amount of force the distal tip of the catheter shaft sees when a known force is being applied to the proximal shaft end of the catheter shaft. With respect to crossability, i.e. the ability to navigate the tip of the catheter shaft across narrow restrictions in the vasculature, the suggested catheter can be moved through a significant stenosis by using the propulsive force applied by the moving balloon by pushing the tip of the catheter shaft with greater force. When a guidewire is further provided in the catheter, the catheter can offer maximal backup support to the guidewire and in addition, the greatest possible pushability, crossability and trackability over the guidewire in narrowed or blocked lumens and passages of the patient's body. More specifically, the catheter can provide backup support to the guidewire in the effort to move it through anatomically difficult areas of lumens of the human body, while—at the same time—it can be accurately repositioned, keeping the balloon inflated and anchored within the lumen. In particular, the trackability of the catheter over the guidewire is high, since once again the propulsive force of the balloon moving close to the tip of the catheter shaft is much greater. This is specifically advantageous in tortuous lumens, where the crossing of the catheter is hindered due to the multiplication of the friction between the catheter shaft and the lumen walls as well as between the catheter shaft and the guidewire caused by the existing tortuosity.

According to an embodiment of the present invention, the flexible moving element is hollow for passing fluid (gas or liquid) therethrough. Here, the surgical tool is advantageously an inflatable balloon that can be inflated by the fluid being delivered to it through the hollow flexible moving element.

The tool-receiving casing advantageously comprises a through opening so that the hollow flexible moving element is in fluid communication with the inflatable balloon being loaded on the tool-receiving casing.

The control handle preferably comprises an opening so that control handle is in fluid communication with the hollow flexible moving element. Thus, a source of fluid (gas or liquid) can be connected to the control handle to pass the fluid through the hollow flexible moving element to the inflatable balloon.

The fluid can for example be air, helium, water or a water solution preferably containing a contrast agent.

When the tool-receiving casing is formed such that it also acts as a control handle and the surgical tool is an inflatable balloon, the tool-receiving casing may advantageously comprise a casing lumen that communicates with the inflatable balloon, more specifically with an inner space of the balloon. Thus, fluid can be provided through the casing lumen to the balloon for inflating it. It is also possible that the catheter comprises either the hollow flexible moving element or the tool-receiving with the casing lumen both for feeding the inflatable balloon with fluid.

Preferably, when the tool-receiving casing also serves as a control handle, the tool-receiving casing is connected to the flexible moving element at one or more connecting positions in the direction of a longitudinal axis of the tool-receiving casing, i.e. over a length of the tool-receiving casing.

To this end, the catheter shaft may comprise apart from the distal shaft wall opening and the proximal shaft wall opening at least one further shaft wall opening. The flexible moving element can thus be attached to the tool-receiving case through the distal and proximal shaft wall openings and the at least one further shaft wall opening of the catheter shaft.

In order to reduce friction losses between the catheter shaft and the tool-receiving casing, the catheter shaft and the tool-receiving casing can preferably be formed such that the catheter shaft and the tool-receiving casing only partially contact each other. Accordingly, in order to reduce friction losses between the catheter shaft and the control casing, the catheter shaft and the control casing can preferably be formed such that the catheter shaft and the tool-receiving casing only partially contact each other.

As mentioned above, the surgical tool of the catheter may be an inflatable balloon. Preferably, the inflatable balloon can be formed such that, when it is in its inflated state, it allows body fluids of the patient to pass by, preferably more easily in one direction than in another direction, most preferably only in one direction. This is in particular advantageous when the catheter is used as a nasogastric catheter and placed inside the oesophagus of the patient. In order for the catheter to be stabilized at a desired position, the balloon has to be inflated so that it is anchored to the oesophagus. The balloon being formed according to this advantageous embodiment of the present invention allows thus saliva to pass by the balloon and not to be accumulated in the oesophagus, while it preferably hinders the reflux of gastric contents to the oesophagus.

The balloon preferably comprises a proximal balloon end, a distal balloon end, and at least one channel extending from the proximal balloon end to the distal balloon end for allowing a flow of fluid therethrough in the inflated state of the balloon.

According to an advantageous embodiment, the at least one channel is formed as a recess in a circumferential area of the balloon. In other words, an outer dimension of a first region of the balloon at a given cross-section is smaller than an outer dimension of a second region of the balloon at the given (same) cross-section. The second region is the region through which the balloon is configured to be in contact with the wall of a body cavity, passage, lumen etc., when the balloon is placed and inflated therein.

The balloon can particularly comprise a main region and at least one secondary region, which protrudes from the main region in the inflated state of the balloon. Thus, the balloon can be anchored to the wall of a body cavity, passage, lumen etc. only via its secondary region(s), while the space(s) between the main region and the secondary region or the secondary regions will allow body fluids to flow along the balloon. For example, the balloon may have four secondary regions, which protrude from the main region and form the shape of a cross, when the balloon is inflated.

According to an alternative advantageous embodiment, the at least one channel extends through an inner space of the balloon.

Regardless of whether the channel is formed as a recess or if extends through an inner space of the balloon, the channel may preferably be formed such that fluid can flow substantially in one direction or only in one direction through the channel.

To this end, a first channel cross-section of the at least one channel is preferably larger than a second channel cross-section of the at least one channel. In particular, the first channel cross-section is arranged at a first channel end, more particularly a proximal channel end, and the second channel cross-section is arranged at a second channel end, more particularly a distal channel end. Preferably, the at least one channel has a tapered form between the first channel cross-section and the second channel cross-section. Additionally or alternatively thereto, a one-way valve is arranged at the first second cross-section or the second channel cross-section.

The surgical tool can also be formed as a cutting device, such as a Fallopian tube scraping wire (curretage wire for uterine curretage) or a blade, e.g. an atherectomy blade, a stent or any other tool that can be used in a surgical procedure, in which a catheter is used, and that can be connected directly or indirectly to the flexible moving element. In the framework of the present invention, the surgical tool may also comprise materials (e.g. a creme, a medical substance) that need to be unloaded within a lumen or cavity of a hollow organ of the human or animal body.

Preferably, the catheter shaft further comprises at least one further lumen for receiving a guidewire and/or passing a fluid therethrough, e.g. for delivering a fluid to a site of interest in the patient's body and/or removing body or other fluids from the patient's body. The at least one further lumen can also be characterized as a guidewire receiving and/or fluid passing lumen. It is also possible that the at least one further lumen comprises at least one guidewire lumen and at least one fluid passing lumen as separate lumens.

The at least one guidewire receiving and/or fluid passing lumen advantageously extends over a whole length of the catheter shaft. In other words, a proximal end and a distal end of the further lumen correspond to a proximal shaft end and a distal end of the catheter shaft, respectively. Alternatively, the at least one guidewire receiving and/or fluid passing lumen may extend a partial length of the catheter shaft.

The flexible moving element receiving lumen(s) and/or the at least one guidewire receiving and/or fluid passing lumen are preferably parallel to each other.

Further, the flexible moving element receiving lumen(s) and/or the at least one guidewire receiving and/or fluid passing lumen may have the same or different shapes and/or sizes.

Preferably, the flexible moving element receiving lumen(s) and/or the at least one guidewire receiving and/or fluid passing lumen each have a cylindrical shape, in particular with a circular cross-section, i.e. the flexible moving element receiving lumen(s) and/or the at least one guidewire receiving and/or fluid passing lumen is/are in particular a circular cylinder. However, the cross-section of the flexible moving element receiving lumen(s) and/or the at least one guidewire receiving and/or fluid passing lumen may have another shape, e.g. the cross-section may be oval.

The catheter may be used with a guidewire arranged in the at least one guidewire receiving and/or fluid passing lumen of the catheter shaft. An assembly of the catheter and the guidewire can be characterized as a catheter assembly in the framework of the present invention.

The guidewire can advantageously be used as a guide for inserting a further catheter, after the guidewire has reached a site of interest inside the human or animal body by using the catheter of the present invention and the catheter of the present invention has been removed from the body of the patient.

The catheter shaft preferably comprises a tip with an opening. The tip of the catheter corresponds to a distal area of the catheter shaft and comprises an opening preferably corresponding to the distal shaft end of the catheter shaft. Further, the catheter shaft comprises a main part, to which the tip is connected. The tip and a portion of the main part of the catheter shaft are located inside the patient's body when the catheter is used, whereas the rest of the main part of the catheter shaft is located outside the patient's body. The cross-section of the tip is preferably tapered.

The catheter shaft may preferably have an internal and/or an external lining of lubricant on an inner surface of the catheter shaft wall defining the flexible moving element receiving lumen(s) and on the outer surface of the catheter shaft wall, respectively. Preferably, the tool receiving casing and/or the control casing has/have an internal lining of lubricant. The lubricant can for example be Teflon. Thus, a friction between the flexible moving element(s) and the flexible moving element receiving lumen(s) and/or between the tool-receiving casing and the catheter shaft and/or between the control casing and the catheter shaft can be reduced.

The surgical tool and/or the flexible moving element and/or the tool-receiving casing and/or the control handle, in particular control casing, advantageously each have two end positions. In the first end position the respective component is closer to the proximal shaft end of the catheter shaft than in the second end position.

In the following, a configuration of the catheter of the present invention in which the surgical tool can be moved by a plurality of flexible moving elements is described.

The catheter may advantageously comprise a plurality of flexible moving elements and a plurality of flexible moving element receiving lumens, wherein the flexible moving element receiving lumens are defined by the catheter shaft wall and wherein each of the flexible moving elements is arranged in one of the flexible moving element receiving lumens and connected to the surgical tool. Only one flexible moving element is arranged in each flexible moving element receiving lumen. Each flexible moving element is arranged in the corresponding flexible moving element receiving lumen in such a way that the flexible moving elements are at least partially circumferentially supported by the catheter shaft wall so that the surgical tool is movable by the plurality of the flexible moving elements. The features and considerations described above with regard to the flexible moving element may advantageously apply to any of the flexible moving elements of the plurality of the flexible moving elements. For example, each flexible moving element may preferably be connected to the already described tool-receiving casing.

Further, the catheter shaft advantageously comprises a plurality of distal shaft wall openings and/or a plurality of proximal shaft wall openings.

In particular, the catheter shaft may comprise a first flexible moving element and a second flexible moving element as well as a first flexible moving element receiving lumen and a second flexible moving element receiving lumen. The first flexible moving element corresponds to the previously described flexible moving element and the first flexible moving element receiving lumen to the previously described flexible moving element receiving lumen. The second flexible moving element receiving lumen is defined by the catheter shaft wall. The second flexible moving element is connected to the tool-receiving casing and arranged in the second flexible moving element receiving lumen in such a way that the second flexible moving element is at least partially circumferentially supported by the catheter shaft wall so that the surgical tool is movable by the second flexible moving element.

Preferably, the tool-receiving casing is connected to the second flexible moving element at one or more connecting positions in the direction of the longitudinal axis of the tool-receiving casing, i.e. over a length of the tool-receiving casing.

Like the first flexible moving element, the second flexible moving element may preferably be formed as a wire or a sheet.

When the surgical instrument is an inflatable balloon, the second flexible moving element may be hollow for passing fluid therethrough for inflating the balloon.

The catheter shaft may comprise a first and a second distal shaft wall opening. The first distal shaft wall opening corresponds to the distal wall shaft opening previously described. The second distal shaft wall opening is formed in a distal circumferential area of the catheter shaft and communicates with the second flexible moving element receiving lumen.

The catheter shaft may comprise a first and a second proximal shaft wall opening. The first proximal shaft wall opening corresponds to the proximal wall shaft opening previously described. The second proximal shaft wall opening is formed in a proximal circumferential area of the catheter shaft and that communicates with the second flexible moving element receiving lumen.

The features previously presented with regard to distal wall shaft opening and/or proximal wall shaft opening previously described may advantageously apply to the second distal wall shaft opening and/or second proximal wall shaft opening, respectively.

It is noted that the formulation “defined by the catheter shaft wall” referring to any of the lumens of the catheter shaft described above means that the respective lumen corresponds to a hollow space of the catheter shaft restricted/limited/bounded by the catheter shaft wall.

The directly preceding description referred to the catheter having one surgical tool that can be moved by a plurality of flexible moving elements.

In the following, a catheter with a plurality of surgical tools is described.

Apart from the previously described surgical tool, the catheter may also comprise at least one further surgical tool that is fixed on the catheter shaft (at least one non-mobile surgical tool) and/or at least one further surgical tool that is movable relative to the catheter shaft (at least one further mobile surgical tool). The features and explanations referring to the design of the previously described surgical tool and its movable arrangement in the catheter advantageously also refer to the at least one further mobile surgical tool.

In other words, the catheter of the present invention may comprise a plurality of surgical tools, from which at least one is a movable surgical tool. A catheter having a plurality of surgical tools comprises in its simplest configuration a first surgical tool and a second surgical tool. Here, the previously described surgical tool corresponds to the first surgical tool. The previously described lumen, flexible moving element, distal piston and proximal piston are a first lumen, a first flexible moving element, a first distal piston and a first proximal piston, respectively. The first surgical tool and the second surgical tool are movable relative to each other. The term “plurality of surgical tools” means “two or more surgical tools”.

According to a preferred embodiment, the second surgical tool is fixed on the surgical tool.

According to an alternative preferred embodiment, the second surgical tool is movable independently from the first surgical tool. In this embodiment, the catheter shaft comprises a second lumen defined by the catheter shaft wall, a second flexible moving element, a second surgical tool connected to the second flexible moving element, a second distal piston and a second proximal piston. The second distal piston and the second proximal piston are arranged in the second lumen and a space between the second distal piston and the proximal piston is filled with an incompressible fluid. The second distal piston is connected to the second flexible moving element. The second flexible moving element is arranged in the catheter shaft in such a way that the second flexible moving element is at least partially circumferentially supported by the catheter shaft wall so that a movement of the second proximal piston in a direction from a proximal shaft end of the catheter shaft to a distal shaft end of the catheter shaft causes a movement of the second surgical tool.

The catheter may preferably comprise a third surgical tool that is fixed on the catheter shaft. The present invention further refers to a method of use of the described catheter in a surgical procedure. The method comprises at least the step of providing a previously described catheter.

These and further details, advantages and features of the present invention will be described based on embodiments of the invention and by taking reference to the accompanying figures. It is shown in

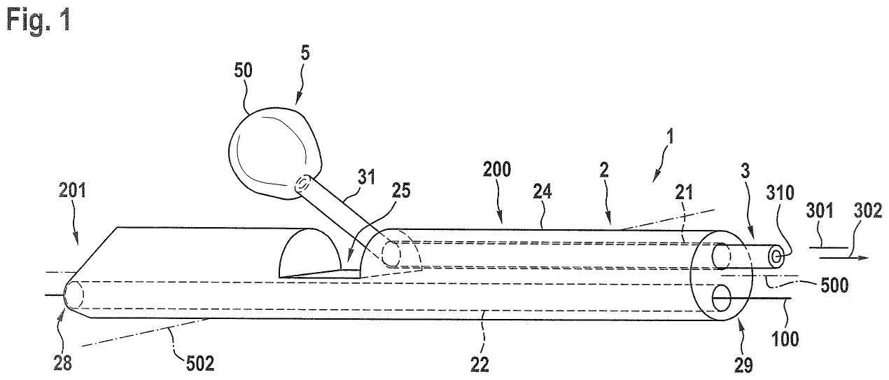

FIG. 1 a simplified perspective view of a catheter according to a first embodiment of the present invention,

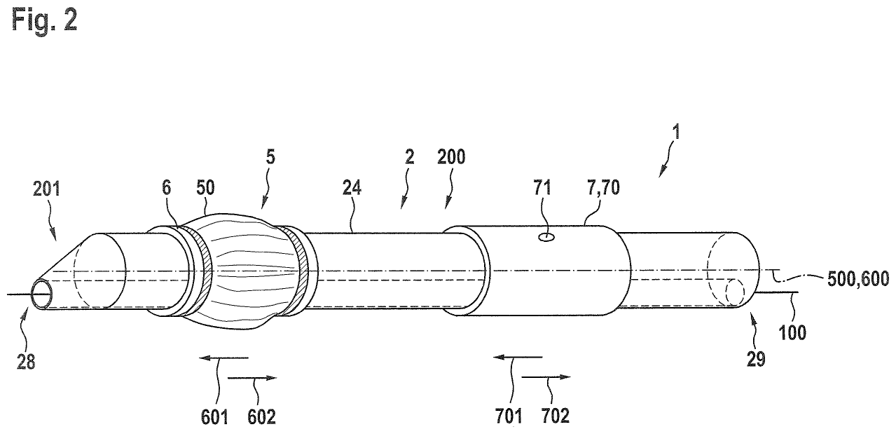

FIG. 2 a simplified perspective view of a catheter according to a second embodiment of the present invention,

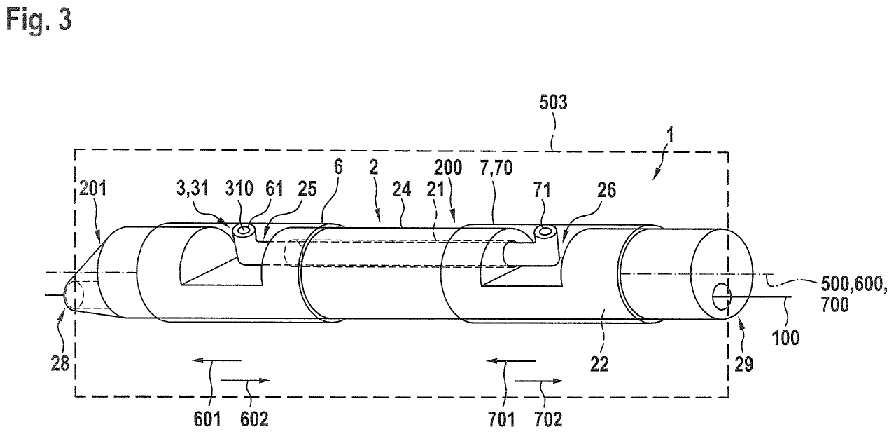

FIG. 3 a simplified perspective view of a part of the catheter according to the second embodiment,

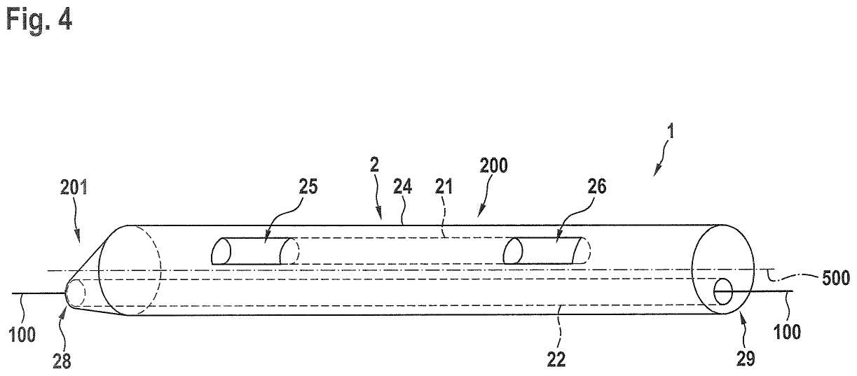

FIG. 4 a simplified perspective view of a part of a catheter according to a third embodiment of the present invention,

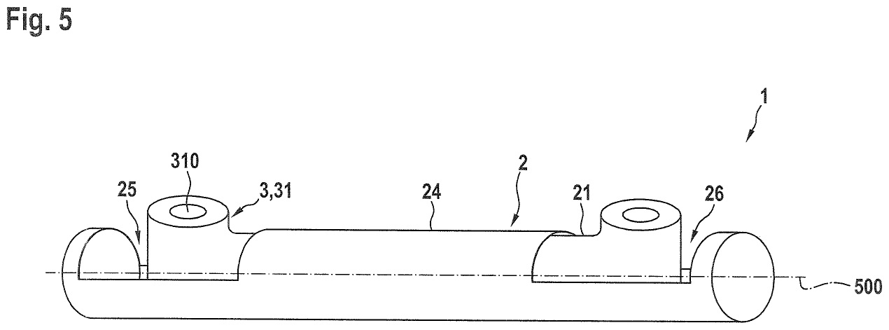

FIG. 5 a simplified perspective view of a part of the catheter according to the third embodiment of the present invention,

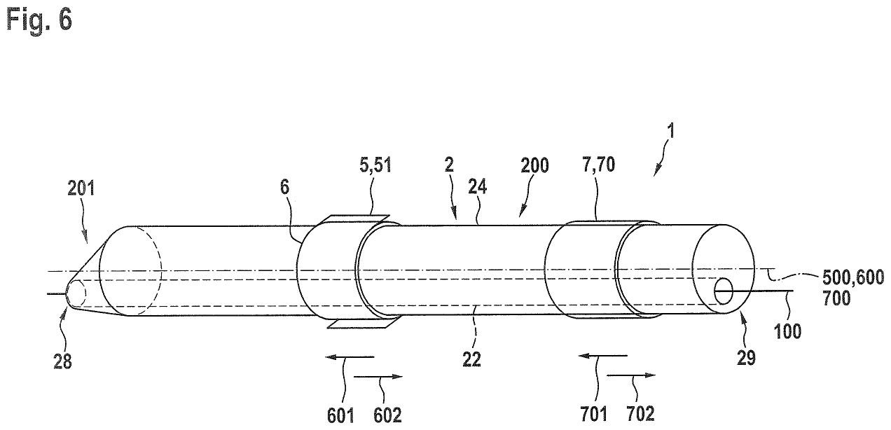

FIG. 6 a simplified perspective view of a catheter according to a fourth embodiment of the present invention,

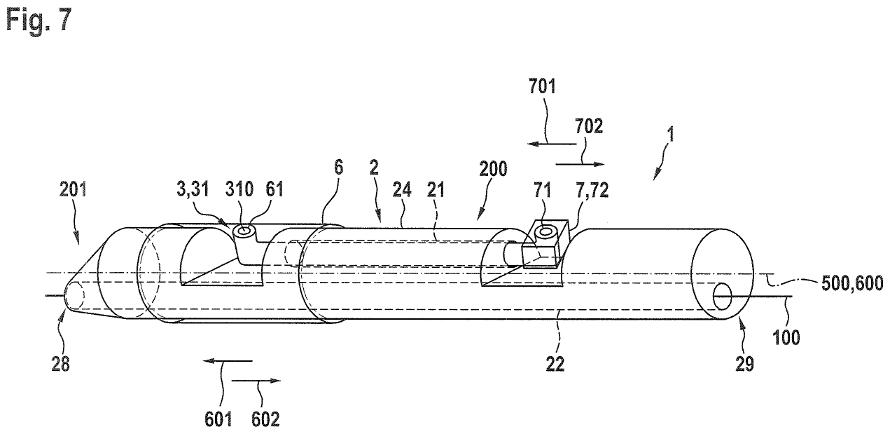

FIG. 7 a simplified perspective view of a part of a catheter according to a fifth embodiment of the present invention,

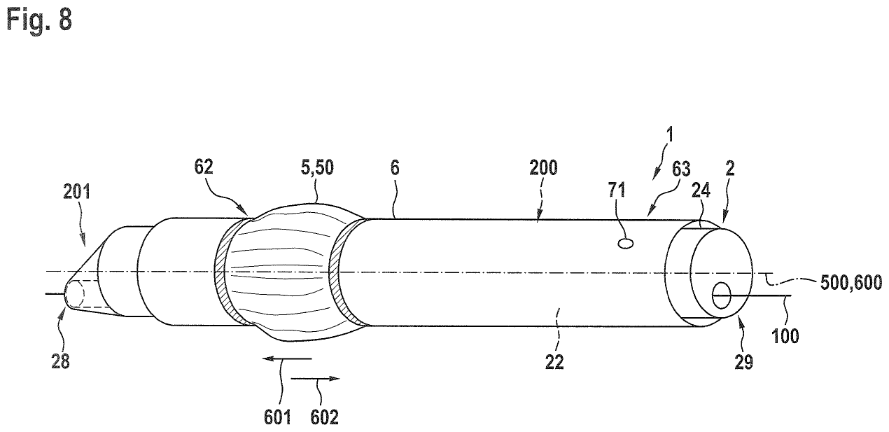

FIG. 8 a simplified perspective view of a catheter according to a sixth embodiment of the present invention,

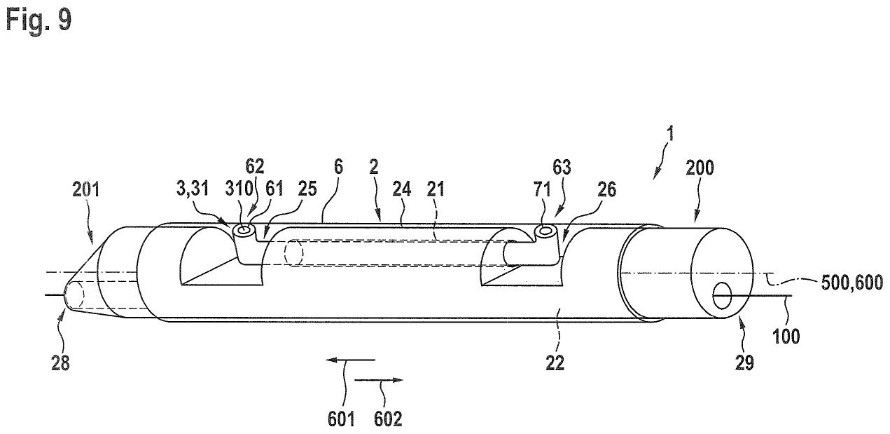

FIG. 9 a simplified perspective view of a part of the catheter according to the sixth embodiment,

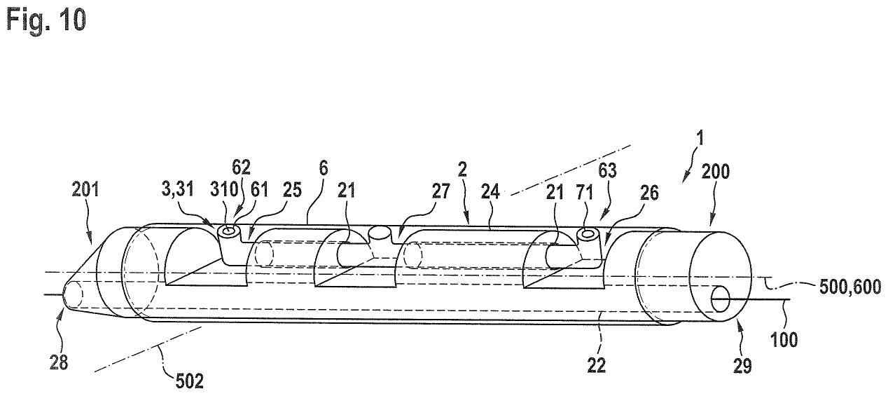

FIG. 10 a simplified perspective view of a part of a catheter according to a seventh embodiment of the present invention,

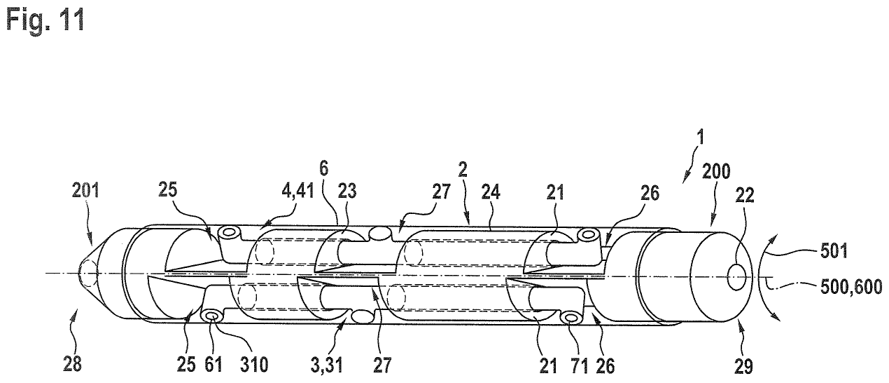

FIG. 11 a simplified perspective view of a part of a catheter according to an eighth embodiment of the present invention,

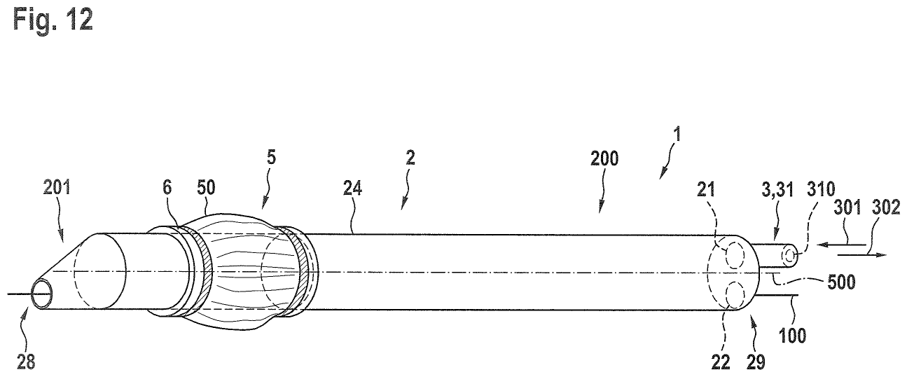

FIG. 12 a simplified perspective view of a part of a catheter according to a ninth embodiment of the present invention,

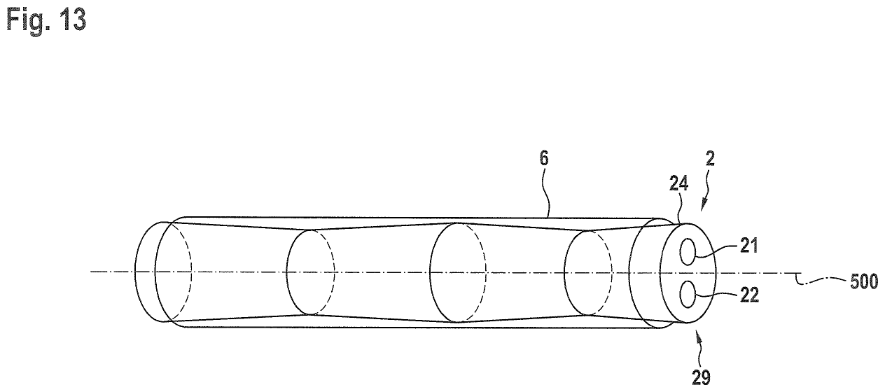

FIG. 13 a simplified perspective view of a part of a catheter according to a tenth embodiment of the present invention,

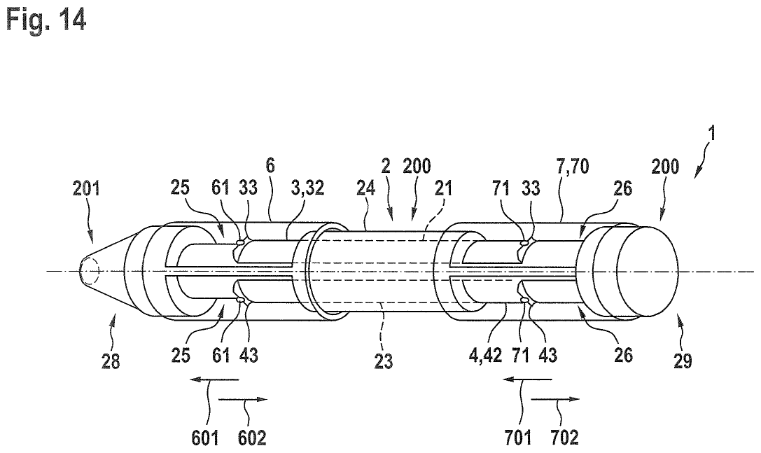

FIG. 14 a simplified perspective view of a part of a catheter according to an eleventh embodiment of the present invention,

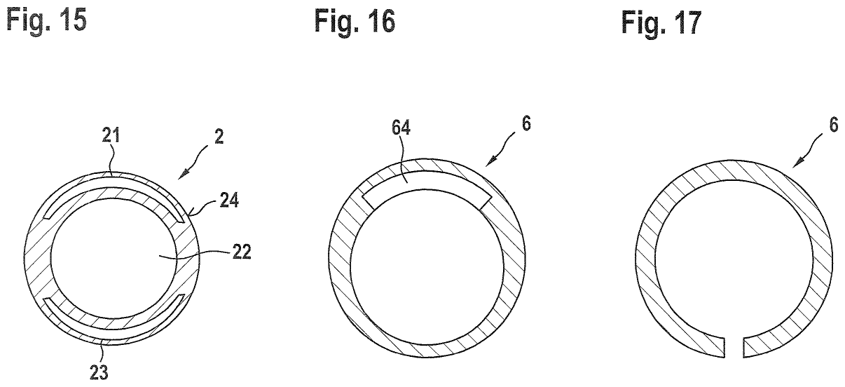

FIG. 15 a cross-section of a catheter shaft of the catheter of FIG. 14,

FIG. 16 a simplified perspective view of a part of the catheter according to a twelfth embodiment of the present invention,

FIG. 17 a cross-section of a part of a catheter according to a thirteenth embodiment of the present invention,

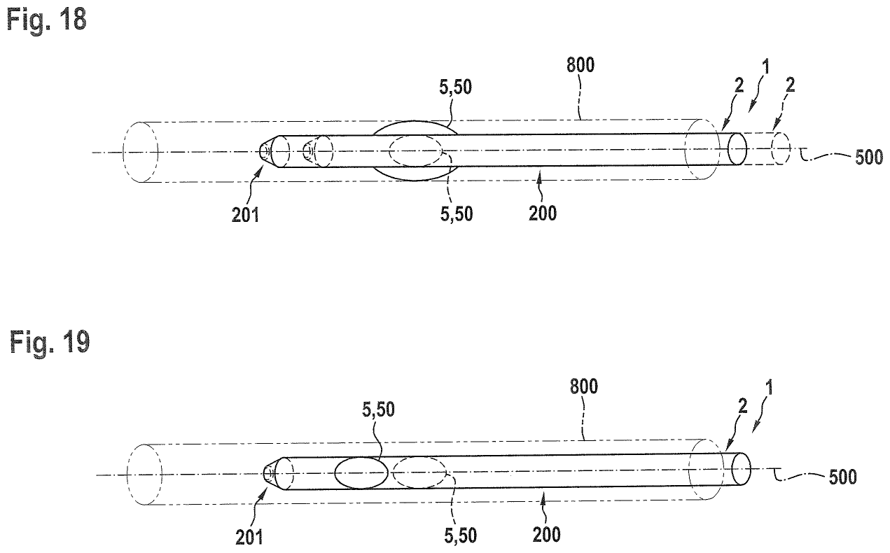

FIG. 18 a simplified side view of a catheter according to present invention in a first and a second state inside a body lumen, and

FIG. 19 a simplified side view of the catheter of FIG. 18 in a third and a fourth state inside the body lumen.

In the following, embodiments and uses of the present invention are presented in detail by taking reference to accompanying FIGS. 1 to 19. Identical or equivalent features and features which act identically or equivalently are denoted with the same reference signs. For the sake of conciseness, a detailed description of the elements and components is not repeated in each case of their occurrence. It is also noted that the length of the catheter is not necessarily drawn to scale.

FIG. 1 shows a perspective view of a catheter 1 according to a first embodiment of the present invention.

As can be seen from said figure, the catheter 1 comprises a catheter shaft 2, a flexible moving element 3 and a surgical tool 5 connected to the flexible moving element 3. The surgical tool 5 is directly connected to the flexible moving element 3 and formed as an inflatable balloon 50.

The catheter shaft 2 comprises a main part 200 and a tip 201 attached to the main part 200 and extends in the direction of a longitudinal axis 500 between a distal shaft end 28 and a proximal shaft end 29.

Further, the catheter shaft 2 comprises a flexible moving element receiving lumen 21 defined by a catheter shaft wall 24. The catheter shaft 2 further comprises a distal shaft wall opening 25, which is formed in a distal circumferential area of the catheter shaft 2 and communicates with the flexible moving element receiving lumen 21. The flexible moving element receiving lumen 21 extends from the proximal shaft end 29 of the catheter shaft 2 to the distal shaft wall opening 25. The distal shaft wall opening 25 is more particularly formed as a through recess in the catheter shaft wall 24 in a direction of a transverse axis 502 of the catheter shaft 2.

When looking further into the structure of the catheter 1, it can be seen that the flexible moving element 3 is formed as a wire 31. The wire 31 is formed as a hollow cylinder and has in particular a channel 310, which is in fluid communication with the inflatable balloon 50. In order to inflate the balloon 50, a fluid—a gas or a liquid—can be delivered to the balloon 50 via the channel 310 of the hollow wire 31.

FIG. 1 shows the balloon 50 in its inflated state and protruding out of the catheter shaft 2. When the balloon 50 is undeployed, i.e. deflated, the balloon 50 can be completely located inside the flexible moving element receiving lumen 21 of the catheter shaft 2. Alternatively, in its deflated state, the balloon 50 can be situated partially inside the flexible moving element receiving lumen 21 and partially inside the distal shaft wall opening 25, or can be situated out of the flexible moving element receiving lumen 21 and inside the distal shaft wall opening 25. It is, however, also possible that the balloon 50 is directly slidably arranged on the catheter shaft 2, in particular the catheter shaft wall 24. For example, the balloon 50 can be formed such that it completely surrounds the catheter shaft 2.

The wire 31 is longitudinally movably, in particular slidably, and rotatably arranged in the flexible moving element receiving lumen 21 such that the wire 31 is circumferentially supportable and/or supported by the catheter shaft wall 24 so that the balloon 50 is movable, in particular pushable, by the wire 31. In other words, due to the circumferential/lateral support provided by the catheter shaft wall 24, the rather flexible wire 31 functions as a stiffer element, when a force, in particular a pushing force towards the distal shaft end 28 of the catheter shaft 2, is applied to it. However, the wire 31 remains capable of being bent together with the catheter shaft 2, when the catheter 1 is steered through a curved pathway in the body of a patient.

In particular, a diameter of the wire 31 is preferably at least 80%, more preferably at least 85%, even more preferably at least 90%, even more preferably at least 95%, most preferably substantially 100%, of a diameter of the flexible moving element receiving lumen 21. Advantageously, a fit between the flexible moving element and the flexible moving element receiving lumen 21 is a clearance fit.

The wire 31 may take a first end position, in which the wire 31 is totally pulled back, and a second end position, in which the wire 31 is totally moved towards the distal shaft end 28 of the catheter shaft 2. In other words, the wire 31 is in particularly retractably and extractably arranged in the flexible moving element receiving lumen 21, wherein the wire 31 is extracted in its second end position and retracted in its first end position. FIG. 1 shows the second end position of the wire 31.

In the present embodiment, a movement of the wire 31 towards the distal shaft end 28 of the catheter shaft 2 denoted by arrow 301 in FIG. 1 results in a movement of the balloon 50 towards the distal shaft end 28 and eventually out of the catheter shaft 2 through the distal shaft wall opening 25. A movement of the wire 31 towards the proximal shaft end 29 of the catheter shaft 2 denoted by arrow 302 in FIG. 1 causes the balloon 50 to be also moved towards the proximal shaft end 29.

It is noted that in both end positions a part of the wire 31 is positioned in the flexible moving element receiving lumen 21 and a part of the wire 31 is located outside of the catheter shaft 2, so that the wire 31 can be held and controlled by the doctor. It is however also possible that the catheter 1 comprises a control handle that is connected to the wire 31, in particular to a proximal end portion or proximal end of the wire 31. The control handle can be formed as a stiff control element, e.g. as a shaft or grip button. Thus, controlling of the movement of the balloon 50 can be effected by manipulating the stiff control element. The control handle is formed such that fluid can be provided through the control handle to the channel 310 of the wire 31 for inflating the balloon 50.

The tip 201 and a portion of the main part 200 of the catheter shaft 2 including the distal shaft wall opening 25 are located inside the patient's body during use of the catheter 1, whereas the rest of the main part 200 of the catheter shaft 2 is located outside the patient's body.

The catheter shaft 2 further comprises a further lumen 22, which is defined by the catheter shaft wall 24. In the catheter 1 of FIG. 1, a guidewire 100 is arranged in the further lumen 22. The catheter 1 together with the guidewire 100 can be characterized as a catheter assembly. The further lumen 22 is in particular parallel to the flexible moving element receiving lumen 21 and extends in a direction parallel to the longitudinal axis 500 of the catheter shaft 2. Both the flexible moving element receiving lumen 21 and the further lumen 22 are formed as circular cylinders.

The catheter 1 of the present embodiment can for example be used as a nasogastric catheter. In such a use, the distal shaft end 28 of the catheter shaft 2 needs to be placed inside the stomach of the patient, while it has to be to ensured that the catheter shaft 2 is prevented from being accidentally withdrawn out of the patient's body. Further, the gastric content needs to be blocked from refluxing to the oesophagus. When using the catheter 1 as a nasogastric catheter, the guidewire 100 is usually not used with the catheter 1.

In order to achieve the aforementioned goals, the catheter 1 is inserted through the nose of the patient past the throat into the oesophagus. The catheter 1 is then forwarded towards the stomach so that the distal shaft end 28 of the catheter shaft 2 passes the gastroesophageal junction and a considerable length of the catheter shaft 2 is located inside the stomach. Then, the doctor pushes the wire 31 towards the distal shaft end 28 of the catheter shaft 2, whereby the deflated balloon 50 is moved towards the distal shaft end 28 and exits the catheter shaft 2 through the distal shaft wall opening 25. Then, the doctor may inflate the balloon 50 by providing fluid to it though the channel 310 of the wire 31. While maintaining the balloon 50 inflated, the doctor pulls the wire 31 towards the proximal shaft end 29 of the catheter shaft 2, thereby causing the balloon 50 to be also pulled towards the proximal shaft end 29 of the catheter shaft 2. However, as it is still inflated, the balloon 50 cannot go back inside the flexible moving element receiving lumen 21 or into the oesophagus and thus, the catheter shaft 2 cannot be accidentally removed from the patient's body.

FIGS. 2 and 3 refer to a catheter 1 according to a second embodiment of the present invention.

With reference to FIG. 2, the catheter 1 of the second embodiment comprises a catheter shaft 2, a tool-receiving casing 6 and a control handle 7.

The tool-receiving casing 6 being formed as a hollow circular cylinder is for receiving and carrying the surgical tool 5, which is also here formed as an inflatable balloon 50. This means that, contrary to the first embodiment, the inflatable balloon 50 is not directly connected to the flexible moving element 3, but fixed on the tool-receiving casing 6. Thus, the inflatable balloon 50 and the tool-receiving casing 6 move together as a single unit, when the tool-receiving casing 6 is moved in the direction of its longitudinal axis 600. The longitudinal axis 600 of the tool-receiving casing 6 coincides with the longitudinal axis 500 of the catheter shaft 2.

The control handle 7, which is used for controlling the movement of the inflatable balloon 50, is formed in the present embodiment as a circular cylindrical control casing 70 and extends in the direction of a longitudinal axis 700. The longitudinal axis 700 of the control casing 70 coincides with the longitudinal axis 500 of the catheter shaft 2.

Both the tool-receiving casing 6 and the control casing 70 are slidably arranged on the catheter shaft 2 and can be moved in the direction of the longitudinal axis 500 of the catheter shaft 2.

In FIG. 3, the inflatable balloon 50 is removed from the catheter 1 and the tool-receiving casing 6 and the control casing 70 are drawn transparent, so that more details of the catheter shaft 1 can be revealed.

In particular, it can be seen that the tool-receiving casing 6 as well as the control casing 70 are fixed to the flexible moving element 3, which is, similarly to the first embodiment, formed as a hollow wire 31 with a channel 310. More specifically, the tool-receiving casing 6 is connected to a distal end of the wire 31, while the control casing 70 is connected to a proximal end of the wire 31. Like in the catheter 1 of the first embodiment, the wire 31 is partially arranged in the flexible moving element receiving lumen 21 such that the wire 31 is circumferentially supportable and/or supported by the catheter shaft wall 24 so that the balloon 50 is movable, in particular pushable, by the wire 31. In particular, a diameter of the wire 31 is preferably at least 80%, more preferably at least 85%, even more preferably at least 90%, even more preferably at least 95%, most preferably substantially 100%, of a diameter of the flexible moving element receiving lumen 21. Advantageously, a fit between wire 31 and the flexible moving element receiving lumen 21 is a clearance fit.

Due to the proposed arrangement, the control casing 70 is configured to control the movement of the inflatable balloon 50 by controlling the tool-receiving casing 6.

Preferably, the flexibility of the wire 31 varies along its length. Particularly, a distal portion and a proximal portion of the wire 31, which comprise the distal end and the proximal end of the wire 31, respectively, have a smaller flexibility than the middle portion of the wire 31. In other words, the connecting portions of the wire 31 with the tool-receiving casing 6 and the control casing 70 have a smaller flexibility than the rest of the wire 31. More specifically, the parts of the wire 31 that are made less flexible than the rest are the parts intended to be situated outside or to come out of the flexible moving element receiving lumen 21 of the catheter shaft 2.

It is noted that in both the first and the second end positions of the wire 31, the wire 31 is arranged in the flexible moving element receiving lumen 21, the distal shaft wall opening 25 and the proximal shaft wall opening 26. For delivering fluid to the wire 31 through the control casing 70 and from the tool-receiving casing 6 to the inflatable balloon 50, the control casing 70 comprises a through opening 71 and the tool-receiving casing 6 a through opening 61, respectively.

As can further be seen from FIG. 3, the catheter shaft 2 comprises apart from the distal shaft wall opening 25 a proximal shaft wall opening 26 formed in a proximal circumferential area of the catheter shaft 2. The proximal shaft wall opening 26 communicates with the flexible moving element receiving lumen 21.

Both the distal shaft wall opening 25 and the proximal shaft wall opening 26 are made as recesses in the catheter shaft wall 24. In particular, the distal shaft wall opening 25 and the proximal shaft wall opening 26 are formed such that a rotation of the wire 31 around its longitudinal axis is possible.

In the present embodiment, the shaft wall openings 25, 26 are formed as recesses extending through the catheter shaft wall 24 in the direction of the transverse axis 502 and allow for a rotation of the wire 31 by up to 90 degrees in each direction with regard to a symmetry plane 503 of the catheter shaft 2.

Further, the tool-receiving casing 6 and the control casing 70 are not only slidably but also rotatably arranged on the catheter shaft 2.

Due to this arrangement, a rotation of the control casing 70 around the longitudinal axis 500 of the catheter shaft 2 results in a rotation of the wire 31 around its longitudinal axis, what in turn results in a rotation of the tool-receiving casing 6 and thus of the balloon 50 too.

For moving the balloon 50, the control casing 70 is moved by the doctor in the direction of the longitudinal axis 500 of the catheter shaft 2. This is denoted by the arrows 701 and 702 in FIG. 1. Particularly, the arrow 701 denotes a movement of the control casing 70 in the direction from the proximal shaft end 29 to the distal shaft end 28, while the arrow 702 denotes a movement of the control handle 70 in the opposite direction. When the control casing 70 is moved in the direction of arrow 701, the tool-receiving casing 6 and thus the balloon 50 fixed thereto is also moved in the direction from the proximal shaft end 29 to the distal shaft end 28. This is denoted by arrow 601. When the control casing 70 is moved in the direction of arrow 702, the tool-receiving casing 6 and thus the balloon 50 is also moved in the direction from the distal shaft end 28 to the proximal shaft end 29. This is denoted by arrow 602.

In the following, a catheter 1 according to a third embodiment of the present invention will be described with reference to FIGS. 4 and 5. FIG. 4 shows a perspective view of a portion of the catheter 1, while FIG. 5 shows a more detailed view of the arrangement of the flexible moving element 3 in the flexible moving element receiving lumen 21 of the catheter 1. As the flexible moving element receiving lumen 21 basically corresponds to a hollow area of the catheter shaft 2 and is defined by the catheter shaft wall 24, the catheter shaft wall 24 is also depicted in FIG. 5. However, in order to allow for a clearer view, only a part of the catheter shaft wall 24 needed to define and graphically represent the flexible moving element receiving lumen 21 is shown in FIG. 5.

The catheter 1 according to according to the third embodiment differs from the catheter 1 of the second embodiment in the way the distal shaft wall opening 25 and the proximal shaft wall opening 26 are formed.

As can be seen from FIGS. 4 and 5, in the catheter 1 according to the third embodiment, the shaft wall openings 25, 26 are formed as elongated openings, so that the wire 31 cannot turn around its longitudinal axis, so that the tool-receiving casing 6 and the control casing 70 can only be moved in the direction of the longitudinal axis 500 of the catheter shaft 2. In other words, the tool-receiving casing 6 and the control casing 70 are not rotatably arranged on the catheter shaft 2 and thus, the balloon 50 being loaded on the tool-receiving casing 6 can also be moved only in the direction of the longitudinal axis 500.

FIG. 6 shows a perspective view of a catheter 1 according to a fourth embodiment of the present invention.

The basic structure of the catheter 1 according to the fourth embodiment corresponds in general to that of the catheter 1 of the third embodiment.

The main difference lies in that the surgical tool 5 loaded on the tool-receiving casing 6 of the catheter 1 of the fourth embodiment is a cutting device 51 instead of an inflatable balloon.

As there is no need to supply fluid to the cutting device 51, the wire 31 is advantageously formed as a solid wire. Within this framework, also the through opening 61 of the tool-receiving casing 6 and the through opening 71 of the control casing 70 of the catheter 1 of the third embodiment can be omitted in the catheter 1 of the fourth embodiment.

For example, the cutting device 51 can be an atheroctomy blade. The catheter 1 can then be used as an endovascular catheter for cutting away an atheroma (atheromatous plaque), i.e. an abnormal accumulation in the inner layer of the wall of a blood of an artery.

In order to achieve this, the catheter 1 will be positioned in the vicinity of the atheroma inside the affected artery. Then, the doctor may move the control casing 70 towards the atheroma, thereby causing the atheroctomy blade to move towards the atheroma and eventually cut it.

The catheter 1 according to the present embodiment can also be used as a urological catheter in a prostatectomy procedure. In this case, the cutting device 51 is a prostate cutter. FIG. 7 shows a perspective view of a portion of a catheter 1 according to a fifth embodiment of the present invention.

The catheter 1 according to the fifth embodiment differs from the catheter 1 according to the second embodiment in that the control handle 7 is not formed as a casing like the control casing 70 but as a control grip button 72.

In order to control the movement of the surgical tool 5, which is also in this embodiment an inflatable balloon 50 (not shown in FIG. 7), the doctor will hold the control grip button 72 in his/her hand and move it in the desired direction.

For supplying the inflatable balloon 50 with fluid for inflation purposes, the control grip button 72 comprises a through opening 71 so that a fluid communication can be established between a fluid source and the balloon 50 via the control grip button 72, the wire 31 and the tool-receiving casing 6.

If the surgical tool 5 is not an inflatable balloon, the wire 31 can be made as a solid wire and the opening 71 in the control grip button 72 can be omitted.

In the following, a catheter 1 according to a sixth embodiment of the present invention will be described with reference to FIGS. 8 and 9.

The catheter 1 of the sixth embodiment mainly differs from the catheter 1 of the second embodiment in that the tool-receiving casing 6 in the catheter 1 of the sixth embodiment is formed such that the tool-receiving casing 6 also serves as a control handle for controlling the movement of the surgical tool 5, in this case the inflatable balloon 50.

In other words, the tool-receiving casing 6 and the control handle 7, more specifically the control casing 70 in the catheter 1 of the second embodiment correspond in functional terms to the tool-receiving casing 6 of the catheter 1 of the sixth embodiment. By using an alternative formulation, the tool-receiving casing 6 of the catheter 1 of the sixth embodiment can be described as the tool-receiving casing 6 and the control casing 70 of the catheter 1 of the second embodiment being integrated with each other or, in other words, made in a single piece.

To serve the purpose of controlling the movement of the balloon 50, the tool-receiving casing 6 has a length that allows it to be arranged partially inside and partially outside the patient's body. In particular, a length of the tool-receiving casing may be more than 70%, preferably more than 80%, and less than 100% of a length of the catheter shaft 2.

As can be seen in particular from FIG. 9, the wire 31 is connected with the tool-receiving casing 6 distally as well as proximally, i.e. at a distal portion 62 as well as at a proximal portion 63 of the tool-receiving casing 6. By providing such a connection between the wire 31 and the tool-receiving casing 6, a movement and a force applied by the doctor to the tool-receiving casing 6 at its proximal portion 63 is transferred to the distal portion 62 of the tool-receiving casing 6 via the wire 31.

FIG. 10 shows a simplified perspective view of a portion of a catheter 1 according to a seventh embodiment of the present invention.

The structure of the catheter 1 according to the seventh embodiment is basically the same with the structure of the catheter 1 according to the sixth embodiment with the difference that the catheter shaft 2 of the catheter 1 of the seventh embodiment comprises in addition to the distal shaft wall opening 25 and the proximal shaft wall opening 26 a further shaft wall opening 27. The further shaft wall opening 27 is arranged between the distal shaft wall opening 25 and the proximal shaft wall opening 26 and formed as a recess through the catheter shaft wall 24 in the direction of the transverse axis 502.

The further shaft wall opening 27 provides a further connection point between the wire 31 and the tool-receiving casing 6. In other words, the wire 31 is connected to the tool-receiving casing 6 (also) via the further shaft wall opening 27.

As can further be seen from FIG. 10, the flexible moving element receiving lumen 21 is not continuously formed, but is interrupted by the further shaft wall opening 27.

By connecting the wire 31 with the tool-receiving casing 6 not only via the distal shaft wall opening 25 and the proximal shaft wall opening 26 but also via the further shaft wall opening 27, a more uniform distribution of a force, which is applied to the tool-receiving casing 6 at its proximal region 63, over the length of the wire 31 can be achieved.

The connecting portions of the wire 31 with the tool-receiving casing 6 may have a smaller flexibility than the rest of the wire 31.

Though the catheter shaft 2 of the catheter 1 according to present embodiment comprises only one further shaft wall opening 27, it is understood that the catheter shaft 2 may comprise a plurality of shaft wall openings 27.

FIG. 11 shows a simplified perspective view of a portion of a catheter 1 according to an eighth embodiment of the present invention.

The catheter 1 according to the eighth embodiment differs from the catheter 1 according to the seventh embodiment in that the catheter shaft 2 of the catheter 1 according to the eighth embodiment comprises a second flexible moving element receiving lumen 23, a second flexible moving element 4, a plurality of distal shaft wall openings 25, a plurality of proximal shaft wall openings 26 and a plurality of further shaft wall openings 27. In particular, the catheter shaft 2 comprises two distal shaft wall openings 25, two proximal shaft wall openings 26 and two further shaft wall openings 27.

It can further be seen from FIG. 11 that one distal shaft wall opening 25, one proximal shaft wall opening 26 and one further shaft wall opening 27 are aligned with each other and form a first row of shaft wall openings. The other distal shaft wall opening 25, the other proximal shaft wall opening 26 and the other further shaft wall opening 27 are also aligned with each other and form a second row of shaft wall openings.

The first row of shaft wall openings is offset from the second row of shaft wall openings in the direction of the longitudinal axis 500 of the catheter shaft 2. In other words, the distal shaft wall openings 25 are offset from each other in the direction of the longitudinal axis 500. The same applies to the proximal shaft wall openings 26 and to the further shaft wall openings 27, respectively.

In addition, the first row of shaft wall openings are preferably offset from the second row of shaft wall openings in a circumferential direction 501 of the catheter shaft 2 by 180 degrees.

The second flexible moving element 4 is preferably formed as a second wire 41 being partially arranged in the second flexible moving element receiving lumen 23. More specifically, the second wire 41 is arranged in the second flexible moving element receiving lumen 23 such that second wire 41 is circumferentially supportable and/or supported by the catheter shaft wall 24 so that a movement of the second wire 41 is transmittable to the surgical tool 5, in this case the inflatable balloon 50. The second wire 41 is preferably, in contrast to the first wire 31, formed as a solid wire. Further, similar to the flexible moving element receiving lumen 21, the second flexible moving element receiving lumen 23 is also not continuously formed but interrupted by the corresponding further shaft wall opening 27.

By providing a connection between the tool-receiving casing 6 and the wires 31, 41 through the first row and the second row of shaft wall openings, a force, which is applied to the tool-receiving casing 6 at its proximal region, can be uniformly distributed over the lengths of the wires 31, 41.

The connecting portions of the wires 31, 41 with the tool-receiving casing 6 may preferably have a smaller flexibility than the rest of the wires 31, 41.

FIG. 12 shows a perspective view of a catheter 1 according to a ninth embodiment of the present invention.

In general, the catheter 1 according to the ninth embodiment differs from that according to the second embodiment in that the catheter 1 according to the ninth embodiment comprises a tool-receiving casing 6, but not a control handle like the control handle 7 of the catheter 1 according to the second embodiment.

To compensate for this, the flexible moving element receiving lumen 21 extends to the proximal shaft end 29 of the catheter shaft 2 in the direction of the longitudinal axis 500. Accordingly, the wire 31 is arranged in the flexible moving element receiving lumen 21 such that a portion of the wire 31, in particular a proximal portion of the wire 31 including the proximal end of the wire 31, is always located outside of the flexible moving element receiving lumen 21.

Thus, in order to control the movement of the inflatable balloon 50, the doctor will hold the proximal portion of the wire 31, thereby stabilizing it, and will move it in the desired direction.

FIG. 13 shows a perspective view of a portion of a catheter 1 according to a tenth embodiment of the present invention.

The catheter 1 according to the tenth embodiment generally corresponds to the catheter 1 of the ninth embodiment.

However, in contrast to the catheter 1 of the ninth embodiment, the catheter 1 according to the tenth embodiment has a catheter shaft 2 and a tool-receiving casing 6 of different shapes.

More specifically, the tool-receiving casing 6 is formed as a hollow circular cylinder, while the catheter shaft 2 preferably comprises at least one region with a varying cross-section in the direction of its longitudinal axis 500. Thus, an inner surface of the tool-receiving casing 6 and an outer surface of the catheter shaft 2 are not complementary to each other.

In particular, the catheter shaft 2 comprises a plurality of frustoconical regions in the present embodiment. The frustoconical regions are specifically extend and are arranged in the direction of the longitudinal axis 500 of the catheter shaft 2 such that the catheter shaft 2 and the tool-receiving casing 6 contact each other only partially when they are moved relative to each other.

It is noted that it is also possible that the catheter shaft 2 comprises regions of another shape and/or with a different arrangement relative to each other, as long as the catheter shaft 2 and the tool-receiving casing 6 do not fully contact each other.

What is achieved by the described configuration of the catheter 1 is that a sliding friction between the catheter shaft 2 and the tool-receiving casing 6 can be reduced.

In the following, a catheter 1 according to an eleventh embodiment of the present invention is described with reference to FIGS. 14 and 15.

As can be seen from FIG. 14, the catheter 1 comprises, like for example the catheter 1 of the second embodiment, a catheter shaft 2, a tool-receiving casing 6, a control casing 70 and an inflatable balloon 50 loaded on the tool-receiving casing 6. The inflatable balloon 50 is omitted from FIG. 14, while the tool-receiving casing 6 and control casing 70 are drawn such that an insight into the inner structure of the catheter shaft 2 is provided.

The tool-receiving casing 6 and the control casing 70 are slidably arranged on the catheter shaft 2.

The catheter 1 further comprises, like for example the catheter 1 of FIG. 11, a first flexible moving element 3 and a second flexible moving element 4, which are partially arranged in a first flexible moving element receiving lumen 21 and a second flexible moving element receiving lumen 23, respectively, and which are each connected to the tool-receiving casing 6 and the control casing 70.

In particular, the first flexible moving element 3 is arranged in the first flexible moving element receiving lumen 21 such that the first flexible moving element 3 is circumferentially supportable and/or supported by the catheter shaft wall 24 so that the tool-receiving casing 6 and consequently the inflatable balloon 50 are movable by the first flexible moving element 3. Accordingly, the second flexible moving element 4 is arranged in the second flexible moving element receiving lumen 23 such that the second flexible moving element 4 is circumferentially supportable and/or supported by the catheter shaft wall 24 so that the tool-receiving casing 6 and consequently the inflatable balloon 50 are also movable by the second flexible moving element 4.

The catheter shaft 2 further comprises two distal shaft wall openings 25 and two proximal shaft wall openings 26. Each of the flexible moving elements 3, 4 is connected to the tool-receiving casing 6 via one distal shaft wall opening 25 and to the control casing 70 via one proximal shaft wall opening 26. To this end, the first and a second flexible moving elements 3, 4 preferably each comprise protrusions 33, 43, in particular at their corresponding distal and proximal ends. Thus, the first and second flexible moving elements 3, 4 each substantially extend in the direction of their corresponding longitudinal axis.

In the catheter 1 of the eleventh embodiment, the first and the second flexible moving elements 3, 4 are formed as curved sheets 32, 42. In order to provide support to the first and the second flexible moving elements 3, 4, namely the curved sheets 32, 42, the first flexible moving element receiving lumen 21 and the second flexible moving element receiving lumen 23 are curved too and have in particular the same form to the first and second flexible moving elements 3, 4, respectively. In particular, a cross-sectional area of the first flexible moving element 3 and the second flexible moving element 4 is preferably at least 80%, more preferably at least 85%, even more preferably at least 90%, even more preferably at least 95%, most preferably substantially 100%, of a cross-sectional area of the first flexible moving element receiving lumen 21 and the second flexible moving element receiving lumen 23. Advantageously, a fit between the flexible moving element and the flexible moving element receiving lumen 21 is a clearance fit.

Thus, the first and the second flexible moving elements 3,4 are at least partially circumferentially supported by the catheter shaft wall 24 so that they can move the tool-receiving casing 6 and thus also the balloon 50 forward.

This can particularly be seen in FIG. 15, which is a cross-sectional view of the catheter shaft 2. From said figure, it can further be seen that the catheter shaft 2 comprises a further lumen 22 for passing fluids therethrough.

For inflating the inflatable balloon 50, the sheets 32, 42 are hollow, while the tool-receiving casing 6 and the control casing 70 comprise two through openings 61 and two through openings 71, respectively. Thereby, a fluid connection between a fluid source and the inflatable balloon 50 can be established via the control casing 70, the sheets 32, 42 and the tool-receiving casing 6.

FIG. 16 shows a cross-section you of a tool-receiving casing 6 of a catheter 1 according to a twelfth embodiment of the present invention.

The catheter 1 according to the twelfth embodiment differs from the catheter 1 according to the eleventh embodiment in that in the catheter 1 according to the twelfth embodiment the tool-receiving casing 6 also serves as the control handle of the catheter 1.

A further difference lies in that the sheets 32, 42 are not hollow in the catheter 1 of the present embodiment. Instead, the inflatable balloon 50 can be inflated by providing fluid thereto via a casing lumen 64 formed in the wall of the tool-receiving casing 6. It thus becomes apparent that the casing lumen 64 extends from a proximal area, in particular a proximal end, to a distal area of the tool-receiving casing 6. In this case, the opening 61 is formed such that it establishes fluid connection between the casing lumen 64 and the inflatable balloon 50.

FIG. 17 shows a cross-sectional view of a tool-receiving casing 6 of a catheter 1 according to a thirteenth embodiment of the present invention.

The catheter 1 of the thirteenth embodiment generally corresponds to the catheter 1 of FIGS. 2 and 3.

The only difference is that the tool-receiving casing 6 of the catheter 1 of the thirteenth embodiment as an open cross-section. Preferably, the tool-receiving casing 6 is formed with an cross-section of its whole length.

Due to this design, the flexibility of the tool-receiving casing 6 is increased compared to a tool-receiving casing 6 with a closed cross-section like the one the catheter of the second embodiment. This enables the catheter 1 to be more easily forwarded through narrow passages and tortuous lumens of the patient's body.

In the following, a general principle of the operation of a catheter 1 with an inflatable balloon 50 according to the present invention will be described with reference to FIGS. 18 and 19. FIG. 18 shows the catheter 1 in a first and a second state inside a body lumen 800, while FIG. 19 shows the catheter 1 in a third and a fourth state inside the body lumen 800.

In the first state, which is represented by dotted lines in FIG. 18, the catheter 1 is positioned at the site of interest inside the body lumen 800, while the inflatable balloon 50 is deflated. Then, the doctor inflates the balloon 50 and operates the catheter 1 such that the catheter shaft 2 is forwarded inside the body lumen 800. In order to achieve this, the doctor will hold the control handle 7 or the tool-receiving casing 6 serving as a control handle stable and push the catheter shaft 2 forward. The state in which the catheter shaft 2 has been moved forward and the balloon 50 is inflated corresponds to the second state of the catheter 1. The second state of the catheter 1 is represented by solid lines in FIG. 18.

Then, the doctor deflates the balloon 50, while the catheter shaft 2 remains in its position. This state corresponds to the third state and is represented by dotted lines in FIG. 19.

Following the deflating step of the balloon 50, the doctor will operate the catheter 1 such that the balloon 50 is moved forward and relative to the catheter shaft 2. This corresponds to the fourth state of the catheter and is shown in FIG. 19 with solid lines.

The process can be repeated until the catheter shaft 2 has been forwarded by the desired distance, e.g. a distance corresponding to the length of a blockage in the body lumen 800.

A more detailed operation of a catheter 1 of the present application will be presented in the following.

Firstly, an endovascular application of the catheter 1 of the present invention will be described in detail. In this case, the catheter 1 may also be characterized as an endovascular catheter.

The surgical tool 5 is preferably an inflatable balloon 50.

More specifically, the catheter 1 of the present invention can be used as a support catheter for providing support to the guidewire 100 when it is being pushed through a narrowing of a blood vessel, e.g. an artery. Such a blockage may be caused by a build-up of a substance called plaque on the inner walls of the artery. Blood flowing over the top of the plaque tends to clot and thus can cause a further increase of the narrowing of the artery, which may eventually result in a blockage of the artery and consequently in a total occlusion.

In order to open an artery narrowed by plaque, a single guidewire can potentially be used. In particular, the guidewire can be fed to the site of the blockage and pushed through the narrowing of the artery. However, if the blockage is large and the plaque is hardened, the use of a single guidewire turns out to be insufficient, as the guidewire bends at its tip due its flexibility and thus cannot be forwarded through the blockage.

This problem can be overcome by using the catheter 1 of the present invention.

At first, the catheter 1 is forwarded to the site in front of the blockage and the guidewire 100 is pushed forward so that it exits through the tip opening of the catheter shaft 2. Due to the support of the guidewire 100 by the catheter shaft 2, the guidewire 100 is able to go through to a first part of the blockage. If the surgeon handling the catheter 1 continues to push the guidewire, the guidewire 100 might be pushed a bit further through the blockage, but will eventually reach a point, at which a further pushing movement by the surgeon will cause the guidewire 100 to start to bend, which in turn will start pushing the catheter shaft 2 against the arterial wall.

As soon as the surgeon realizes that the catheter shaft 2 tends to be pushed against the arterial wall, the surgeon stops pushing the guidewire 100 any further and inflates the balloon 50 until it is anchored to the arterial wall. This provides a further support to the guidewire 100, so that the guidewire 100 can now be pushed further through the blockage. Again, when the guidewire 100 is pushed to the extent that a certain length is outside of the catheter shaft 2, the support of the catheter shaft 2 will not anymore be enough to prevent the guidewire 100 from bending, if the guidewire 100 is pushed further. As this bending would occur inside the blockage, this could cause some damage to or ever perforate the artery.

At this stage, the surgeon immobilizes the component of the catheter that serves a control handle and controls the movement of the balloon 50, while the balloon 50 is inflated, and pushes the catheter shaft 2 forward. By doing so, the force with which the surgeon pushes the catheter shaft 2 is transferred to the balloon 50, which is actually what pushes the catheter shaft 2 through the blockage. As the catheter shaft 2 is moved forward, the guidewire 100 is almost completely covered again by the catheter shaft 2. The reason for the catheter shaft 2 being able to be moved forward through the blockage is that the balloon 50 is very close to the site of the blockage and thus the force with which the catheter shaft 2 is pushed towards the blockage is high enough so that the catheter shaft 2 can overcome the resistance through the blockage.

Then, the surgeon pushes the guidewire 100 forward once more. The guidewire, being again supported by the catheter shaft 2, manages to go further through the blockage, until the guidewire 100 starts to bend again, when the length of the guidewire 100 being not anymore covered by the catheter shaft 2 has again reached a certain length.

The surgeon then repeats the procedure until the catheter shaft 2 manages to go through the blockage.

If the blockage has a considerable length, it could be that the distance by which the catheter shaft 2 can be moved with respect to the balloon 50, when the balloon 50 is inflated, is not enough to cover all the length of the blockage by just alternately moving the guidewire 100 and the catheter shaft 2 as described above.

In this case, when the catheter shaft 2 has been moved by the maximum possible distance after the execution of the aforementioned steps, the surgeon deflates the balloon, while stabilizing the catheter shaft 2, and moves it forward by moving forward the component of the catheter 1 that controls the balloon 50. After having brought the deflated balloon 50 to the new desired position, the surgeon inflates the balloon 50 so that it gets anchored to the arterial wall at the new desired position.

Then, the steps of alternating movements of the guidewire 100 and the catheter shaft 2 as presented above are repeated, thereby allowing the extra supported guidewire 100 to be pushed further and exit the blockage.

The surgeon may now deflate the balloon 50 and pull the catheter 1 of the present invention out of the patient, while holding the guidewire 100 in place. At this point, only the guidewire 100 is located inside the patient's body. In particular, the tip of the guidewire 100 is located in a healthy part of the artery past the blockage. The guidewire 100 can then serve as the guide for bringing a catheter having a stent to the site where the blockage was located.

To summarize, the procedure for opening the blockage in the artery comprises alternate forward movements of the guidewire 100 and the catheter shaft 2, while the balloon is in its inflated state, so that the guidewire 100 is supported by the catheter shaft 2 when the guidewire 100 is being moved forward. Furthermore, in case the blockage has a considerable length, the balloon 50 will be deflated, moved forward and anchored at a new position closer or inside to the blockage in order to provide support to the guidewire 100 and the catheter shaft 2.

It is noted that the balloon 50 is usually located outside the blockage, more specifically in front of the blockage, throughout the whole procedure and that it can progressively be forwarded closer (even inside) to the blockage site.

By having the balloon 50 inflated and anchored within the artery, the movement of the balloon 50 in relation to the catheter shaft 2 is translated into the movement of the catheter shaft 2 to the balloon 50 and the artery—at the same time. Thus, the catheter shaft 2 can move within the artery accurately and powerfully both forwards and backwards, because of the inflated-anchored balloon 50. The balloon 50 may have a movement range of a few centimetres close to the tip of the catheter shaft 2. This practically means that the propulsive force that moves the catheter shaft 2 is applied close to its tip, achieving maximal pushability of the tip and therefore of the catheter shaft 2 as a whole, since the propulsion force is applied from a distance closer to the tip.

In the following, a nasogastric application of the catheter 1 of the present invention will be described. In this case, the catheter 1 may also be characterized as a nasogastric catheter and can be used for gaining access to the stomach and its contents, enabling drainage of the gastric contents, decompressing the stomach, obtaining a specimen of the gastric contents, or introduce a passage into the gastrointestinal tract.

The surgical tool 5 is preferably an inflatable balloon 50.

According to a first technique, the doctor inserts the catheter 1 through the nose of the patient, past the throat and down into the stomach and places it at a position, at which gastric contents can be drained. At this stage, the balloon 50 is in its deflated state.

When it is ensured that the catheter shaft 2 is placed at the desired position, the doctor inflates the balloon 50. The doctor then moves the inflated balloon 50 towards the proximal shaft end of the catheter shaft 2 by manipulating the component of the catheter 1 that controls the balloon 50, while keeping the catheter shaft 2 stable.

When the balloon 50 reaches the area of the gastrooesophageal junction and contacts the upper wall of the stomach, it cannot move any further, as due to its inflated state the balloon has a larger cross-sectional area than the oesophagus. At this moment, the doctor feels in his/her hand that the catheter shaft 2 tends to move towards the stomach. The doctor immediately stops moving the balloon 50 any further and stabilizes it at this position. Following these steps, it can be ensured that the catheter shaft 2, more specifically its distal shaft end 28, is securely placed at the desired position inside the gastric contents, as the inflated balloon 50 prevents the catheter from being accidentally pulled out of the nose of the patient. More importantly, the inflated balloon 50 isolates the stomach from the oesophagus. Thereby, the gastric contents cannot enter the oesophagus and there is no risk of an aspiration pneumonia.

If it is preferred that the balloon 50 is anchored to the oesophagus, the doctor, after completing the aforementioned steps, deflates the balloon 50 and pulls it towards the oesophagus, while maintaining the position of the catheter shaft 2. When the balloon 50 is located inside the oesophagus, the doctor may inflate the balloon 50 again, so that it is anchored to the oesophagus.

According to a second technique, the doctor feeds the catheter 1 through the nose, past the throat into the oesophagus of the patient, so that, depending on the patient's height, the catheter shaft 2 has entered the stomach but not reached a position yet, at which the gastric contests can be drained. In other words, the distal shaft end 28 of the catheter shaft 2 positioned such that it is not located inside the gastric contents. At this stage, the balloon 50 is deflated and located in the oesophagus of the patient.

Then, the doctor inflates the balloon 50, which is now anchored to the oesophagus. While maintaining the balloon 50 inflated and the component of the catheter 1 that controls the movement of the balloon 50 stabilized so that the balloon 50 cannot be moved, the doctor pushes the catheter shaft 2 further inside the stomach of the patient. In addition to pushing the catheter shaft 2, the doctor may rotate the catheter shaft 2 in order to achieve the desired position in the patient's stomach, i.e. that the distal shaft end 28 of the catheter shaft 2 is positioned within the gastric contents. When the desired position has been reached, the doctor stabilizes the balloon 50, thereby also stabilizing the catheter 1 at the desired position.

In case the inflated balloon 50 has to remain inside the patient's oesophagus for a longer period of time, what may cause complications to the patient, or if any complications have already arisen due to the presence of the inflated balloon 50 in the patient's oesophagus, the doctor may change the position of the balloon 50 after some time or as soon as the complications have been observed, respectively. To this end, the doctor will deflate the balloon 50 and move it to a new position, while maintaining the position of the catheter shaft 2. After that, the doctor will inflate the balloon 50 again, thereby stabilizing it at its new position to the oesophagus. By doing so, already existing complications can be dealt with or future complications can be avoided, as the balloon 50 does not press the oesophagus at the same position for the whole time that the catheter 1 is placed inside the patient. The particular advantage of the catheter 1 of the present invention is that the repositioning of the balloon 50 can be done without having to first remove the catheter 1 out of the patient and/or having to use a new catheter of a different length.

According to a third technique, the surgeon introduces the catheter 1 through the nose, past the throat, the oesophagus and the stomach and into the duodenum of the small intestine. At this stage, the balloon 50 is in a deflated state and located inside the stomach. The balloon 50 is then inflated and moved proximally, while the catheter shaft 2 is stabilized. Similar to the second technique, when the balloon 50 reaches the area of the gastrooesophageal junction and contacts the upper wall of the stomach, it cannot move any further, as it has a larger cross-sectional area than the oesophagus due to its inflated state. At this moment, the doctor feels in his/her hand that the catheter shaft 2 tends to move towards the stomach. The surgeon immediately stops moving the balloon 50 any further and stabilizes it at this position. In case the surgeon wants to move the balloon to a new position within the oesophagus, the balloon 50 has first to be deflated and then moved proximally past the gastrooesophageal junction. When the new desired position inside the oesophagus has been reached, the balloon 50 is inflated again. Alternatively, the balloon 50 could be placed inside the oesophagus from the beginning. This is in particular advantageous, if the catheter shaft 2 has to be forwarded in a subsequent step into the small intestine further than the duodenum.

A further nasogastric application of the catheter 1 of the present application is described below.

More specifically, the catheter 1 of the present invention can be used in the case of an oesophageal stricture, i.e. a narrowing or tightening of the oesophagus that causes swallowing difficulties. The stricture usually occurs at the distal end of the oesophagus.

Firstly, the doctor introduces the catheter 1 through the nose and past the throat into the oesophagus of patient until the tip of the catheter shaft 2 reaches the site of the stricture. During this step, the balloon 50 of the catheter 1 is in its deflated state and located inside the oesophagus. At this point, the doctor inflates the balloon 50, which is thus anchored to the oesophagus, thereby providing support to the tip of the catheter shaft 2. If, instead, the doctor continued to push the catheter shaft 2 forward with the balloon 50 being deflated the catheter shaft 2 not being able to pass through the stricture would start to bend and coil up inside the oesophagus.

Following the inflating step of the balloon 50, the doctor pushes the catheter shaft 2 forward while stabilizing the component of the catheter 1 that controls the movement of the balloon 50. Thereby, the doctor transfers the force with which the catheter shaft 2 is pushed to the inflated balloon 50, which is actually that what pushes the catheter shaft 2 through the stricture. The fact that the catheter shaft 2 manages to go through the stricture relies on that the balloon 50 is anchored to the oesophagus close to the stricture and thus the force with which the balloon 50 pushes the catheter shaft 2 is sufficiently high to overcome the resistance of the stricture.

The catheter shaft 2 is pushed in this state until it has entered the stomach, more specifically until a considerable length of the catheter shaft 2 is situated inside the stomach. While maintaining the catheter shaft 2 stable, the doctor now deflates the balloon 50 and pushes it forward by manipulating the component of the catheter 1 that controls its movement.

When the balloon 50 is located inside the stomach, the doctor inflates it again. Then, the doctor pulls the inflated balloon 50 towards the proximal shaft end of the catheter shaft 2, until the doctor feels a resistance due to the balloon 50 touching the upper walls of the stomach. While keeping the component of the catheter 1 that controls the movement of the balloon 50 stable, the doctor now pushes the catheter shaft 2 forward. Again, the balloon 50 is what forces the catheter shaft 2 to be forwarded further inside the stomach. When the desired position is reached, the doctor stops pushing the catheter shaft 2 and stabilizes the balloon over the catheter 1.

Finally, a urological use of the catheter 1 of the present invention will now be described. In this case, the catheter 1 may also be characterized as a urological catheter.

More specifically, the catheter 1 of the present invention can be used in the case of a urethral stricture. A urethral stricture involves scarring that narrows the tube that carries urine out the urethra and thus restricts the flow of urine from the bladder, thereby increasing the risk for the occurrence of a variety of medical problems in the urinary tract, including inflammation or infection. In particular, a urethral stricture can occur due to enlargement of the prostate (prostate hyperplasia).

In order to gain access to the urinary bladder, the doctor may insert the catheter 1 of the present invention into the urethra of the patient up to the stricture in the area of the prostate.

The procedure to be followed in order for the catheter 1 to enter the urinary bladder is similar to that followed in the case of an oesophagus stricture. At the end of the procedure, the distal shaft end 28 of the catheter shaft 2 is located inside the urinary bladder, while the catheter shaft 2 cannot accidentally be removed out of the patient's body due to the balloon 50 being inflated inside the urinary bladder and touching the lower walls thereof.

It is noted that the depicted and described features and further properties of the invention's embodiments can arbitrarily be isolated and recombined without leaving the gist of the present invention.

In addition to the foregoing description of the present invention, for an additional disclosure explicit reference is taken to graphic representation of FIGS. 1 to 19.

### LIST OF REFERENCE SIGNS

1 catheter
2 catheter shaft
3 flexible moving element (first flexible moving element)
4 second flexible moving element
5 surgical tool
6 tool-receiving casing
7 control handle
8 first piston
9 second piston
21 (first) flexible moving element receiving lumen
22 further lumen
23 second flexible moving element receiving lumen
24 catheter shaft wall
25 distal shaft wall opening
26 proximal shaft wall opening
27 further shaft wall opening
28 distal shaft end
29 proximal shaft end
31 wire
32 sheet
33 protrusion
41 wire
42 sheet
43 protrusion
50 balloon
51 cutting device
61 opening
62 distal region
63 proximal region
64 casing lumen
70 control casing
71 opening
72 control grip button
100 guidewire
200 main part
201 tip
301 arrow
302 arrow
310 channel (first channel)
410 channel (second channel)
500 longitudinal axis
501 circumferential direction
502 transverse axis
503 symmetry plane
600 longitudinal axis
601 arrow
602 arrow
700 longitudinal axis
701 arrow
702 arrow
800 body lumen

1. A catheter, comprising:
a flexible moving element,
a catheter shaft that extends along a longitudinal axis and comprises a flexible moving element receiving lumen defined by a catheter shaft wall, and
a surgical tool connected to the flexible moving element,
wherein the flexible moving element arranged in the flexible moving element receiving lumen in such a way that the flexible moving element is at least partially circumferentially supported by the catheter shaft wall so that a movement of the flexible moving element in a direction from a proximal shaft end of the catheter shaft to a distal shaft end of the catheter shaft causes a movement of the surgical tool.

2. The catheter of claim 1, wherein the catheter shaft comprises:
a distal shaft wall opening that is formed in a distal circumferential area of the catheter shaft and communicates with the flexible moving element receiving lumen and/or a proximal shaft wall opening that is formed in a proximal circumferential area of the catheter shaft and communicates with the flexible moving element receiving lumen,
and/or
wherein the flexible moving element receiving lumen extends from the proximal shaft end of the catheter shaft or wherein a proximal shaft end of the flexible moving element receiving lumen is spaced apart from the proximal shaft end of the catheter shaft.

3. The catheter of claim 1, wherein one or more of:
at least the flexible moving element portion that is movably arranged in the flexible moving element receiving lumen between a first end position and a second end position of the flexible moving element C has a cross-sectional area over at least a part of its whole length that is at least 80%, more preferably at least 85%, even more preferably at least 90%, even more preferably at least 95%, most preferably substantially 100%, of a cross-sectional area of the flexible moving element receiving lumen,
wherein at least the flexible moving element portion that is movably arranged in the flexible moving element receiving lumen between a first end position and a second end position of the flexible moving element has over at least a part of its whole length the same shape with the flexible moving element receiving lumen,
wherein at least said flexible moving element portion and the flexible moving element receiving lumen are formed as circular cylinders,
wherein at least said flexible moving element portion has over at least a part of its whole length a diameter that is at least 80%, more preferably at least 85%, even more preferably at least 90%, even more preferably at least 95%, most preferably substantially 100%, of a diameter of the flexible moving element receiving lumen, and
wherein the flexible moving element is arranged in the flexible moving element receiving lumen in such a way that the flexible moving element portion that is movably arranged in the flexible moving element receiving lumen between a first end position and a second end position of the flexible moving element has over at least a part of its whole length a shape that is complementary to a shape of the flexible moving element receiving lumen.

4. The catheter of claim 1, wherein the surgical tool is directly connected to the flexible moving element, and/or wherein the surgical tool is longitudinally movably arranged on the catheter shaft, wherein the surgical tool is directly arranged on the catheter shaft.

5. The catheter of claim 1, further comprising a tool-receiving casing that is longitudinally movably arranged on the catheter shaft, wherein the tool-receiving casing is connected to the flexible moving element and wherein the surgical tool arranged on the tool-receiving casing.

6. The catheter of claim 5, wherein the tool-receiving casing is formed such that the tool-receiving casing also serves as a control handle for controlling the movement of the surgical tool.

7. The catheter of claim 6, wherein the surgical tools is an inflatable balloon and the tool-receiving casing comprises a casing lumen that communicates with the inflatable balloon for inflating the balloon.

8. The catheter of claim 1, further comprising a control handle that is connected to the flexible moving element and configured to control the movement of the surgical tool, wherein the control handle is preferably formed as a control casing that is longitudinally movably, arranged on the catheter shaft and connected to the tool-receiving casing, by the flexible moving element.

9. The catheter of claim 5, wherein the tool-receiving casing and/or the control casing rotatably arranged on the catheter shaft, wherein the tool-receiving casing and the catheter shaft are threadably engaged with each other.

10. The catheter of claim 1, wherein the flexible moving element is formed as a wire or a sheet.

11. The catheter of claim 1, wherein the surgical instrument is an inflatable balloon and the flexible moving element is hollow for passing fluid therethrough for inflating the balloon.

12. The catheter of claim 5, wherein the tool-receiving casing is connected to the flexible moving element at one or more connecting positions in the direction of a longitudinal axis of the tool-receiving casing.

13. The catheter of claim 5, wherein at least a portion of the tool-receiving casing comprises an open cross-section.

14. The catheter of claim 5, wherein the flexible moving element is a first flexible moving element and the flexible moving element receiving lumen is a first flexible moving element receiving lumen, and wherein the catheter further comprises a second flexible moving element receiving lumen defined by the catheter shaft wall and a second flexible moving element, wherein the second flexible moving element is connected to the tool-receiving casing and is arranged in the second flexible moving element receiving lumen in such a way that the second flexible moving element is at least partially circumferentially supported by the catheter shaft wall so that the surgical tool is also movable by the second flexible moving element in the direction from the proximal shaft end of the catheter shaft to the distal shaft end of the catheter shaft,
wherein:
the tool-receiving casing is connected to the second flexible moving element at one or more connecting positions in the direction of a longitudinal axis of the tool-receiving casing, and/or
the second flexible moving element is formed as a wire or a sheet, and/or the surgical instrument is an inflatable balloon and the second flexible moving element is hollow for passing fluid therethrough for inflating the balloon.

15. The catheter of claim 1, wherein the catheter shaft comprises at least one further lumen for receiving a guidewire and/or passing fluid therethrough.
